(19) United States
(12) Patent Application Publication
(10) Pub. No.: US 2009/0156825 A1
(43) Pub. Date: Jun. 18, 2009

(54) Fluorescent compounds that bind to histone deacetylase

(76) Inventors: Richard W. Heidebrecht, JR., Brookline, MA (US); Astrid M. Kral, Holliston, MA (US); Thomas A. Miller, Brookline, MA (US)

Correspondence Address: MERCK AND CO., INC, P O BOX 2000, RAHWAY, NJ, 07065-0907, US

(21) Appl. No.: 12/313,799

(22) Filed: Nov. 25, 2008

Related U.S. Application Data
(60) Provisional application No. 61/004,303, filed on Nov. 26, 2007.

(51) Int. Cl. C07D 213/78

(52) U.S. Cl. 546/306

(57) ABSTRACT

The present invention relates to a novel class of fluorescent compounds that bind to histone deacetylases. The fluorescent compounds can be used to determine binding association and dissociation rates of histone deacetylase inhibitors via fluorescence polarization.

### FIELD OF THE INVENTION

The present invention relates to a novel class of fluorescent compounds that bind to histone deacetylases. The fluorescent compounds can be used to determine binding association and dissociation rates of histone deacetylase inhibitors via fluorescence polarization.

### BACKGROUND OF THE INVENTION

The inhibition of HDACs can repress gene expression, including expression of genes related to tumor suppression. Inhibition of histone deacetylase can lead to the histone deacetylase-mediated transcriptional repression of tumor suppressor genes. For example, inhibition of histone deacetylase can provide a method for treating cancer, hematological disorders, such as hematopoiesis, and genetic related metabolic disorders. More specifically, transcriptional regulation is a major event in cell differentiation, proliferation, and apoptosis. There are several lines of evidence that histone acetylation and deacetylation are mechanisms by which transcriptional regulation in a cell is achieved (Grunstein, M., Nature, 389: 349-52 (1997)). These effects are thought to occur through changes in the structure of chromatin by altering the affinity of histone proteins for coiled DNA in the nucleosome. There are five types of histones that have been identified. Histones H2A, H2B, H3 and H4 are found in the nucleosome, and H1 is a linker located between nucleosomes. Each nucleosome contains two of each histone type within its core, except for H1, which is present singly in the outer portion of the nucleosome structure. It is believed that when the histone proteins are hypoacetylated, there is a greater affinity of the histone to the DNA phosphate backbone. This affinity causes DNA to be tightly bound to the histone and renders the DNA inaccessible to transcriptional regulatory elements and machinery.

The regulation of acetylated states occurs through the balance of activity between two enzyme complexes, histone acetyl transferase (HAT) and histone deacetylase (HDAC).

The hypoacetylated state is thought to inhibit transcription of associated DNA. This hypoacetylated state is catalyzed by large multiprotein complexes that include HDAC enzymes. In particular, HDACs have been shown to catalyze the removal of acetyl groups from the chromatin core histones.

It has been shown in several instances that the disruption of HAT or HDAC activity is implicated in the development of a malignant phenotype. For instance, in acute promyelocytic leukemia, the oncoprotein produced by the fusion of PML and RAR alpha appears to suppress specific gene transcription through the recruitment of HDACs (Lin, R. J. et al., Nature 391:811-14 (1998)). In this manner, the neoplastic cell is unable to complete differentiation and leads to excess proliferation of the leukemic cell line.

U.S. Pat. Nos. 5,369,108, 5,932,616, 5,700,811, 6,087,367 and 6,511,990, disclose hydroxamic acid derivatives useful for selectively inducing terminal differentiation, cell growth arrest or apoptosis of neoplastic cells. In addition to their biological activity as antitumor agents, these hydroxamic acid derivatives have recently been identified as useful for treating or preventing a wide variety of thioredoxin (TRX)-mediated diseases and conditions, such as inflammatory diseases, allergic diseases, autoimmune diseases, diseases associated with oxidative stress or diseases characterized by cellular hyperproliferation (U.S. application Ser. No. 10/369,094, filed Feb. 15, 2003). Further, these hydroxamic acid derivatives have been identified as useful for treating diseases of the central nervous system (CNS) such as neurodegenerative diseases and for treating brain cancer (See, U.S. application Ser. No. 10/273,401, filed Oct. 16, 2002).

The inhibition of HDAC by the hydroxamic acid containing compound suberoylanilide hydroxamic acid (SAHA) disclosed in the above referenced U.S. patents, is thought to occur through direct interaction with the catalytic site of the enzyme as demonstrated by X-ray crystallography studies (Finnin, M. S. et al., Nature 401:188-193 (1999)). Further, hydroxamic acid derivatives such as SAHA have the ability to induce tumor cell growth arrest, differentiation and/or apoptosis (Richon et al., Proc. Natl. Acad. Sci. USA, 93:5705-5708 (1996)). These compounds are targeted towards mechanisms inherent to the ability of a neoplastic cell to become malignant, as they do not appear to have toxicity in doses effective for inhibition of tumor growth in animals (Cohen, L. A. et al., Anticancer Research 19:4999-5006 (1999)).

Many assays are used to identify HDAC inhibitors and reveal kinetics of binding of the HDAC inhibitors. However, in cases where the enzymology does not follow simple Michaelis-Menton assumptions, the assays become extremely labor intensive and lose accuracy. Thus, it is important to develop improved assays that minimize the time for completion and provide data that is of high quality.

### SUMMARY OF THE INVENTION

The present invention relates to a novel class of fluorescent compounds that bind to histone deacetylases. The fluorescent compounds can be used to determine binding association and dissociation rates of histone deacetylase inhibitors via fluorescence polarization.

The present invention thus relates to compounds represented by Formula I and II and pharmaceutically acceptable salts, solvates and hydrates thereof, as detailed herein.

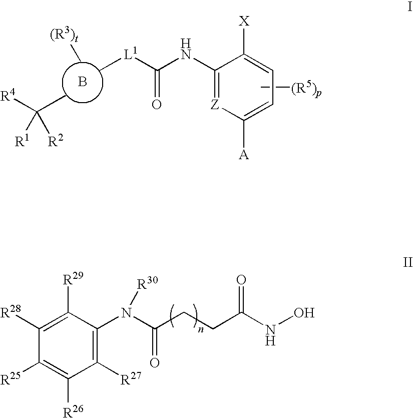

### BRIEF DESCRIPTION OF THE DRAWINGS

FIG. 1 shows the determination of K_{d}^{app} values of the FITC-labeled compounds.

(A) Determination of K_{d}^{app} as a function of time. The curve shows the binding isotherms at 1 minute (grey) and 1 hour (solid).
(B) Replot of K_{d}^{app} values (from (A)) vs. time. At steady-state, the plateau defines K_{d}. A 1-phase exponential fit is shown in grey, a 2-phase exponential fit in solid. FITC-SAHA fits 1-phase exponential curve quite well, whereas COMPOUND 2 requires a 2-phase exponential curve in order to obtain a good fit.

FIG. 2 shows the determination of Kᵢ values and mechanism of binding of test compounds.

A replot of the Inflection Point (IP) values determined for a test compound at different times. At steady-state the plateau defines Kᵢ^{app,}* which when converted by Equation 4, gives the Kᵢ* value.

### DETAILED DESCRIPTION OF THE INVENTION

The present invention relates to a novel class of fluorescent compounds that bind to histone deacetylases. The fluorescent compounds can be used to screen compounds for potency; can be used to demonstrate that potential HDAC inhibitors affect binding of the fluorescent compound known to bind the active site either by binding the active site itself or via an allosteric mechanism; can be used to understand the kinetic mechanism of binding leading to a prediction and better understanding of the time course of inhibitor action in cells as well as dosing schedule in vivo. Based on the mechanism of binding, the actual Kᵢ value of the inhibitors can be determined efficiently. The fluorescence polarization assay using these compounds is superior to kinetic assays with regards to ease of assay set-up and a reduced requirement of material.

Fluorescence polarization theory arises from the observation that when a fluorescently labled molecule is excited with plane polarized light, it emits light that has a degree of polarization that is inversely proportional to its molecular rotation.

A fluorophore is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Examples of fluorophores include but are not limited to the fluorophore in fluorescein, BODIPY TMR dye, BODIPY TR dye, Cascade Blue, Cascade Yellow, Dapoxyl Dyes, Marina Blue, Lucifer yellow, Pacific Blue dyes, Oregon Green 488 dye, Oregon Green 514 dye, NODIPY FL dye, tetramethylrhodamine, rhodamine, X-Rhodamine, rhodamine 6G, rhodamine B, rhodamine 123, Rhodamine Red, Rhodamine Green, Rhodol Green, sulforhodamine 101, Texas Red, coumarin, hydroxycoumarin, aminocoumarin, methoxycoumarin, cyanine, Alexa Fluor dyes, DyLight 549 and DyLight 633.

Alexa Fluor dyes include by are not limited to Alexa Fluor 350, Alexa Fluor 430, Alexa Fluor 488, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750 from Molecular Probes, Inc.

Reactive moieties that attach the fluorophore to the HDAC inhibitor include but are not limited to amine-reactive succinimidyl ester, amine reactive isothiocyanate, thiol-reactive maleimide, thiol-reactive epoxide, amine-reactive acetyl azide and iodoacetamides. Many commercially available reagents that contain the fluorophore and the reactive moiety are available through Molecular Probes Inc. Examples of commercially available reagents include but are not limited to:

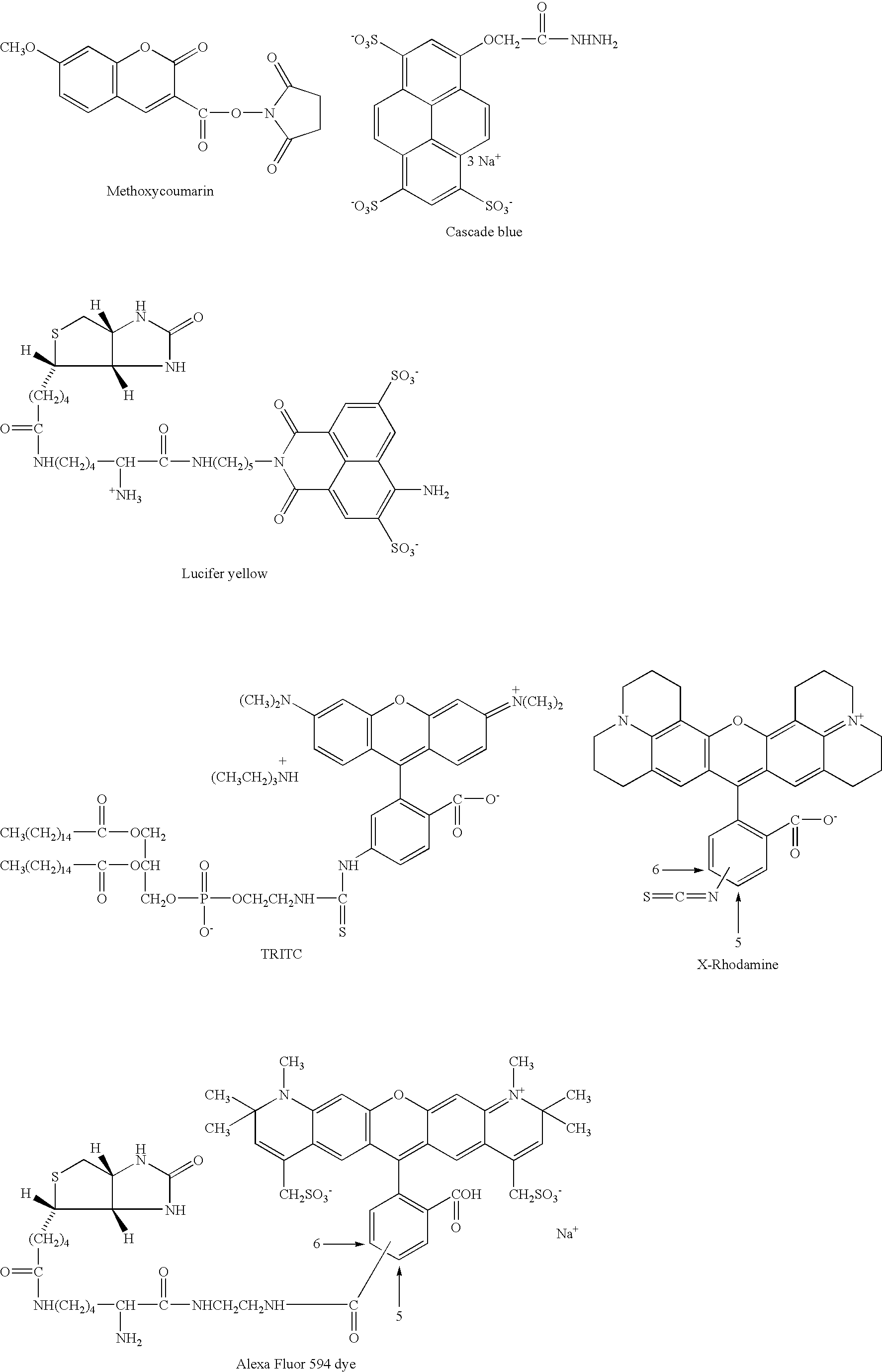

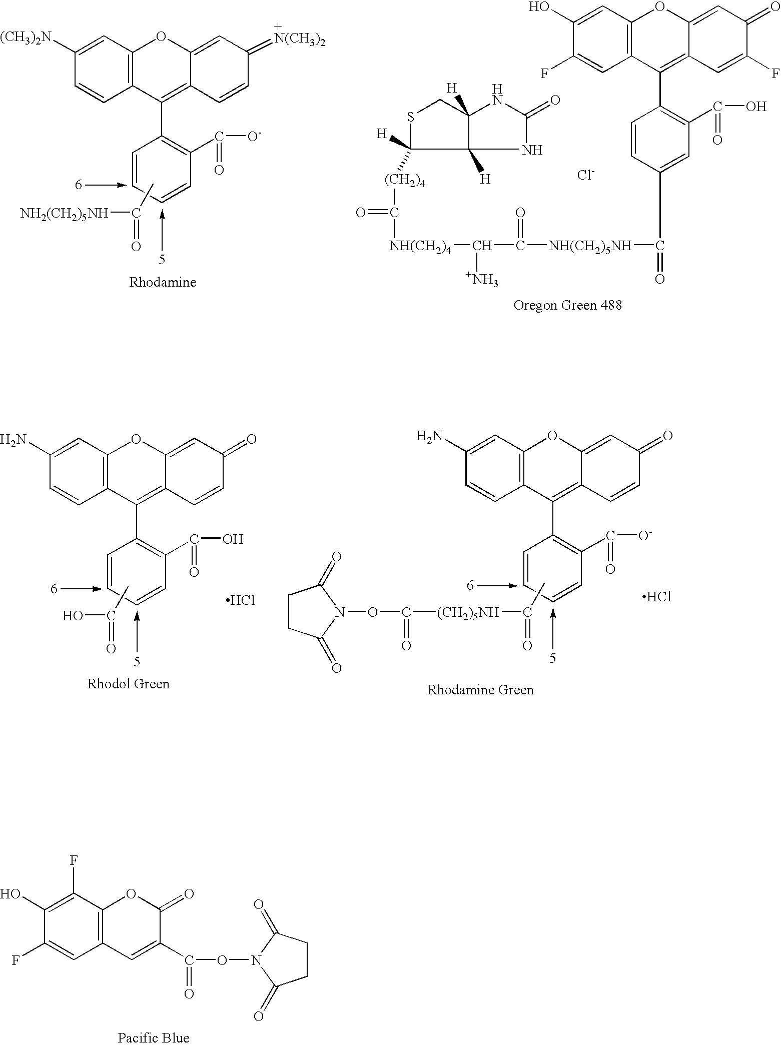

Once the HDAC inhibitor reacts with the reactive moiety on the fluorophore, it forms an HDAC inhibitor attached to a fluorophore through a linker.

In one embodiment, the linker between the fluorophore and the HDAC inhibitor is selected from

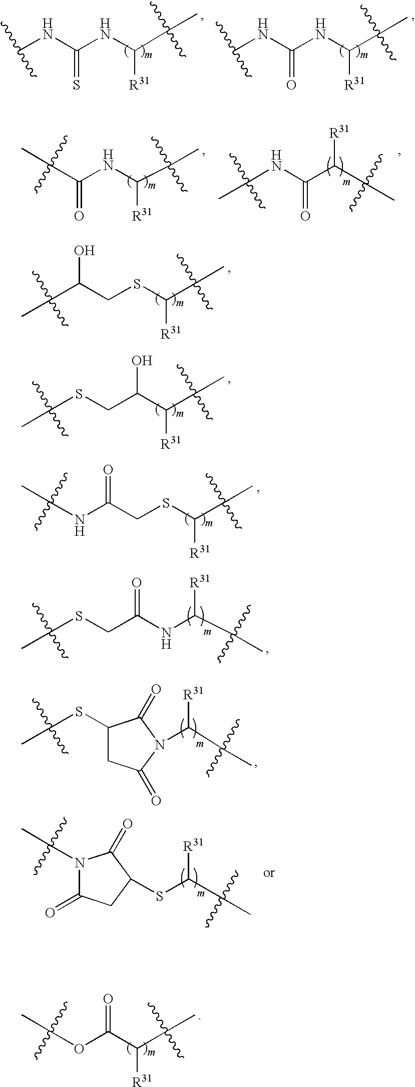

### Compounds

The present invention provides a compound represented by the following structural Formula

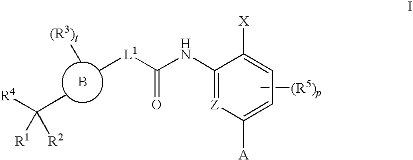

wherein A is aryl, heteroaryl or H, wherein the aryl or heteroaryl is optionally substituted with halo, methyl, methoxy, amino, hydroxyl or halomethyl;

R¹ and R² are independently selected from H, OH, halo, NH₂, C₁-C₄ alkyl, or C₁-C₄ alkoxy;

R³ is independently selected from H, OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino;

R⁴ is selected from —NR⁶R⁷;

R⁵ is independently selected from H, OH, NH₂, nitro, CN, amide, carboxyl, C₁-C₂ alkoxy, C₁-C₂ alkyl, C₁-C₂ haloalkyl, C₁-C₂ haloalkyloxy, C₁-C₂ hydroxyalkyl, C₁-C₂ alkenyl, C₁-C₂ alkyl-C(═O)O—, C₁-C₂ alkyl-C(═O)—, C₁-C₂ alkynyl, halo, hydroxyalkoxy, C₁-C₂ alkyl-NHSO₂—, C₁-C₂ alkyl-SO₂NH—, C₁-C₂ alkylsulfonyl, C₁-C₂ alkylamino or di(C₁-C₂)alkylamino;

R⁶ is independently selected from H or C₁-C₄ alkyl;

R⁷ is selected from —(CR^{a}₂)ₛC(O)(CR^{a}₂)_{q}R¹², or —(CR^{a}₂)ₛC(O)O(CR^{a}₂)_{q}R¹²;

R¹² is selected from C₁-C₄ alkyl, C₃-C₆ cycloalkyl, heteroaryl, aryl or heterocyclic, wherein the alkyl, cycloalkyl, heteroaryl, heterocyclic or aryl is attached to a fluorophore through a linker, and optionally substituted OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino, aryl, heterocyclic or cycloalkyl;

R^{a} is independently selected from H or C₁-C₄ alkyl;

Ring B is aryl or heteroaryl;

p is 1, 2, 3 or 4;

s and q are independently 0, 1, 2, 3, or 4;

L¹ is (CH₂)ᵣ, ethenyl or cyclopropyl, wherein r is 0, 1 or 2;

X is OH or NH₂;

Z is C or N;

or a stereoisomer or pharmaceutically acceptable salt thereof.

In one embodiment, A is

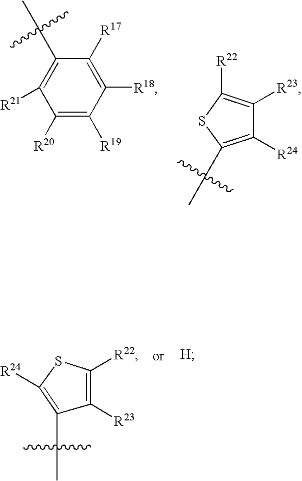

R¹ and R² are independently selected from H, OH, halo, NH₂, C₁-C₄ alkyl, or C₁-C₄ alkoxy;

R³ is H;

R⁴ is —NR⁶R⁷;

R⁵ is H;

R⁶ is selected from H or C₁-C₄ alkyl;

R⁷ is —C(O)O(CR^{a}₂)_{q}R¹²;

R¹² is selected from aryl or heteroaryl; wherein the aryl or heteroaryl is attached to a fluorophore through a linker, and optionally substituted with OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—C₁-C₇ alkylsulfonyl, C₁-C₇ alkylamino or di(C₁-C₇)alkylamino, aryl, heterocyclic or cycloalkyl;

R¹⁷ and R²¹ are independently selected from hydrogen or fluoro;

R¹⁸, R¹⁹ or R²⁰ are independently selected from hydrogen, halo, methyl, methoxy or halomethyl;

R²², R²³ and R²⁴ are independently selected from hydrogen, methyl, amino, hydroxyl or halo;

R^{a} is independently H or C₁-C₄ alkyl;

Ring B is aryl or heteroaryl;

q is independently 0, 1 or 2;

L¹ is a bond;

X is NH₂;

or a stereoisomer or pharmaceutically acceptable salt thereof.

In another embodiment under the foregoing embodiments, A is

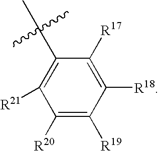

In another embodiment under the foregoing embodiments, R¹ and R² are H; R^{a} is H; R⁶ is H, and q is 1.

In one embodiment under the foregoing embodiments, the fluorophore is selected from the fluorophore in fluorescein, BODIPY TMR dye, BODIPY TR dye, Cascade Blue, Cascade Yellow, Dapoxyl Dyes, Marina Blue, Lucifer yellow, Pacific Blue dyes, Oregon Green 488 dye, Oregon Green 514 dye, NODIPY FL dye, tetramethylrhodamine, rhodamine, X-Rhodamine, rhodamine 6G, rhodamine B, rhodamine 123, Rhodamine Red, Rhodamine Green, Rhodol Green, sulforhodamine 101, Texas Red, coumarin, hydroxycoumarin, aminocoumarin, methoxycoumarin, cyanine, Alexa Fluor dyes, DyLight 549 and DyLight 633. In another embodiment, the fluorophore is

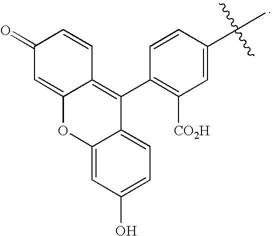

In one embodiment under the foregoing embodiments, the linker is

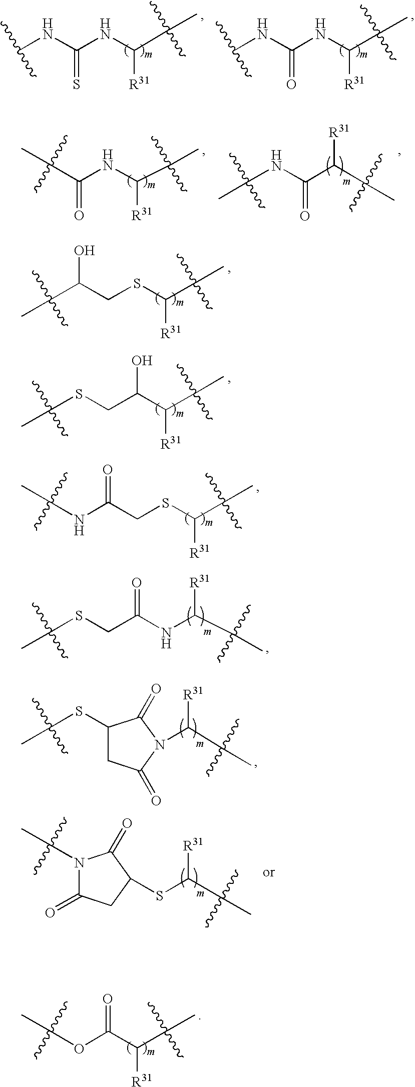

wherein R³¹ is H or C₁-C₄ alkyl;

m is 0, 1 or 2.

In one embodiment for the above embodiments, the linker is

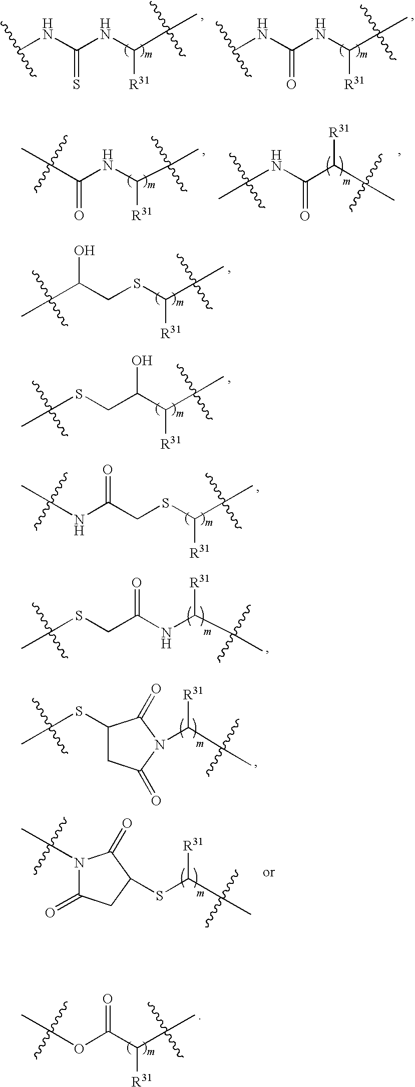

In one embodiment, the linker is

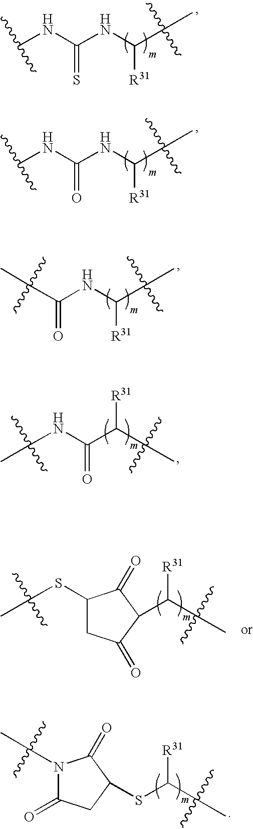

In one embodiment, the linker is

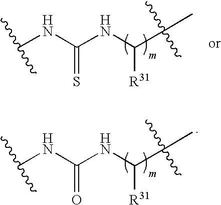

In another embodiment, the linker is

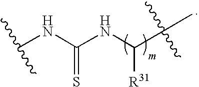

In one embodiment under the foregoing embodiments, R³¹ is H or C₁-C₄ alkyl; m is 0, 1 or 2.

In one embodiment under the foregoing embodiments, R³¹ is H; m is 1.

The present invention also provides a compound represented by the following structural Formula

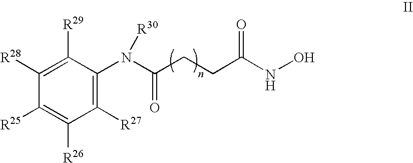

Wherein,
R²⁵ is F-L-, wherein L is a linker, and F is a fluorophore;
R²⁶ to R²⁹ is independently selected from H, C₁-C₄ alkyl, CN, azido, C₁-C₄ cyanoalkyl, nitro, halo, C₁-C₄ haloalkyl, amino, amide, carboxyl, C₁-C₄ alkoxycarbonyl, C₁-C₄ alkylaminocarbonyl, hydroxyl, C₁-C₄ alkoxy, aryl-C₁-C₄-alkoxy, C₁-C₄ haloalkyloxy, C₁-C₄ hydroxyalkyl, C₁-C₄ alkenyl, C₁-C₄ alkyl-C(═O)O—, C₁-C₄ alkyl-C(═O)—, C₁-C₄ alkynyl, hydroxyalkoxy, C₁-C₄ alkyl-NHSO₂—, C₁-C₄ alkyl-SO₂NH—, C₁-C₄ alkylsulfonyl, C₁-C₄ alkylamino or di(C₁-C₄)alkylamino;
R³⁰ is selected from H or C₁-C₄ alkyl;
n is 4, 5, 6, 7 or 8;

or a stereoisomer or pharmaceutically acceptable salt thereof.

In one embodiment under the above embodiments under Formula II, the fluorophore is selected from the fluorophore in fluorescein, BODIPY TMR dye, BODIPY TR dye, Cascade Blue, Cascade Yellow, Dapoxyl Dyes, Marina Blue, Lucifer yellow, Pacific Blue dyes, Oregon Green 488 dye, Oregon Green 514 dye, NODIPY FL dye, tetramethylrhodamine, rhodamine, X-Rhodamine, rhodamine 6G, rhodamine B, rhodamine 123, Rhodamine Red, Rhodamine Green, Rhodol Green, sulforhodamine 101, Texas Red, coumarin, hydroxycoumarin, aminocoumarin, methoxycoumarin, cyanine, Alexa Fluor dyes, DyLight 549 and DyLight 633.

In another embodiment, the fluorophore is

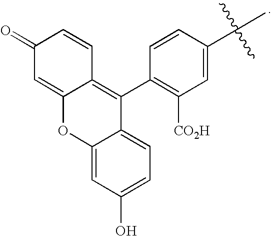

In another embodiment under the above embodiments under Formula II the linker is

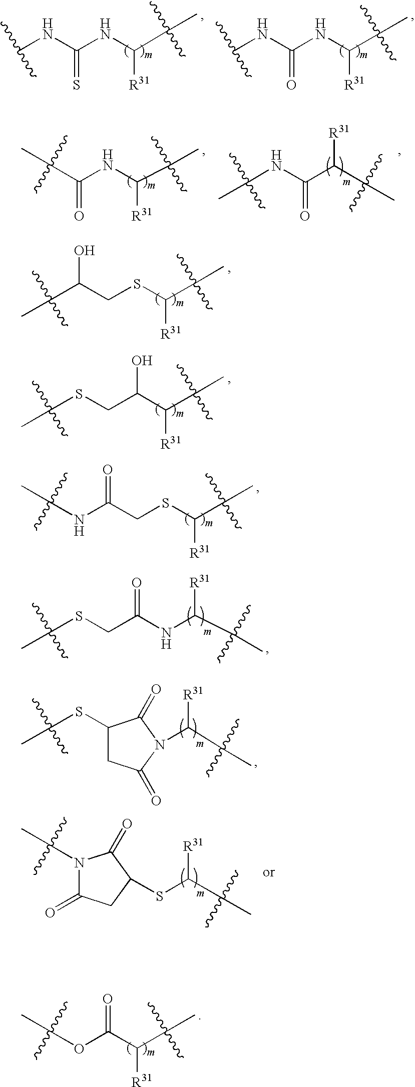

wherein R³¹ is H or C₁-C₄ alkyl;

m is 0, 1 or 2.

In one embodiment, the linker is

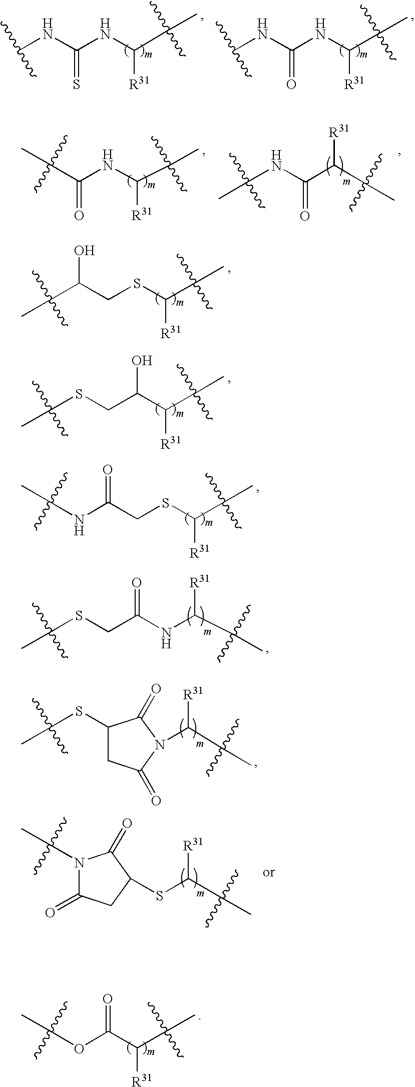

In another embodiment, the linker is

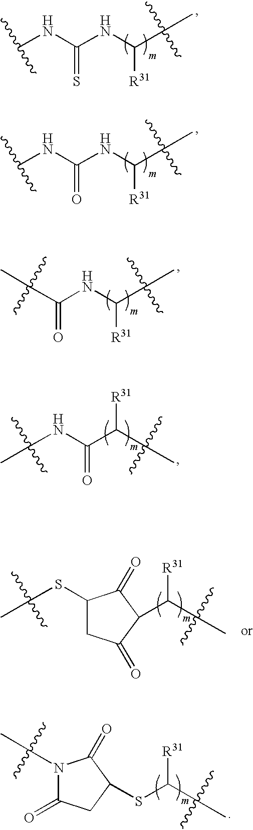

In one embodiment, the linker is

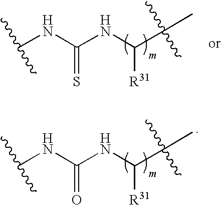

In another embodiment, the linker is

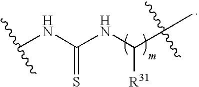

Specific examples of the compounds of the instant invention include:
5-{[({4-[({[(4-{[(4-aminobiphenyl-3-yl)amino]carbonyl}benzyl)amino]carbonyl}oxy)methyl]benzyl}amino)carbonothioyl]amino}-2-(6-hydroxy-3-oxo-3H-xanthen-9-yl)benzoic acid;
5-({[(4-{[8-(hydroxyamino)-8-oxooctanoyl]amino}benzyl)amino]carbonothioyl}amino)-2-(6-hydroxy-3-oxo-3H-xanthen-9-yl)benzoic acid;
5-{[({4-[({[(4-{[(2-aminophenyl)amino]carbonyl}benzyl)amino]carbonyl}oxy)methyl]benzyl}amino)carbonothioyl]amino}-2-(6-hydroxy-3-oxo-3H-xanthen-9-yl)benzoic acid.
   or the pharmaceutically acceptable salt or stereoisomer thereof.

### Chemical Definitions

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylaryl”, “alkylcycloalkyl” and “alkylheterocyclyl” the term “alkyl” refers to the alkyl portion of the moiety and does not describe the number of atoms in the aryl and heteroaryl portion of the moiety. In an embodiment, if the number of carbon atoms is not specified, the “alkyl” of “alkylaryl”, “alkylcycloalkyl” and “alkylheterocyclyl” refers to C₁-C₁₂ alkyl and in a further embodiment, refers to C₁-C₆ alkyl.

The term “cycloalkyl” means a monocyclic saturated or unsaturated aliphatic hydrocarbon group having the specified number of carbon atoms. The cycloalkyl is optionally bridged (i.e., forming a bicyclic moiety), for example with a methylene, ethylene or propylene bridge. The bridge may be optionally substituted or branched. The cycloalkyl may be fused with an aryl group such as phenyl, and it is understood that the cycloalkyl substituent is attached via the cycloalkyl group. For example, “cycloalkyl” includes cyclopropyl, methyl-cyclopropyl, 2,2-dimethyl-cyclobutyl, 2-ethyl-cyclopentyl, cyclohexyl, cyclopentenyl, cyclobutenyl and so on.

In an embodiment, if the number of carbon atoms is not specified, “alkyl” refers to C₁-C₁₂ alkyl and in a further embodiment, “alkyl” refers to C₁-C₆ alkyl. In an embodiment, if the number of carbon atoms is not specified, “cycloalkyl” refers to C₃-C₁₀ cycloalkyl and in a further embodiment, “cycloalkyl” refers to C₃-C₇ cycloalkyl. In an embodiment, examples of “alkyl” include methyl, ethyl, n-propyl, i-propyl, n-butyl, t-butyl and i-butyl.

The term “alkylene” means a hydrocarbon diradical group having the specified number of carbon atoms. For example, “alkylene” includes —CH₂—, —CH₂CH₂— and the like. In an embodiment, if the number of carbon atoms is not specified, “alkylene” refers to C₁-C₁₂ alkylene and in a further embodiment, “alkylene” refers to C₁-C₆ alkylene.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

“Aryl” is intended to mean any stable monocyclic, bicyclic or tricyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In one embodiment, “aryl” is an aromatic ring of 6 to 14 carbons atoms, and includes a carbocyclic aromatic group fused with a 5- or 6-membered cycloalkyl group such as indan. Examples of carbocyclic aromatic groups include, but are not limited to, phenyl, naphthyl, e.g. 1-naphthyl and 2-naphthyl; anthracenyl, e.g. 1-anthracenyl, 2-anthracenyl; phenanthrenyl; fluorenonyl, e.g. 9-fluorenonyl, indanyl and the like. A carbocyclic aromatic group is optionally substituted with a designated number of substituents, described below.

The term heteroaryl, as used herein, represents a stable monocyclic, bicyclic or tricyclic ring of up to 7 atoms in each ring, wherein at least one ring is aromatic and contains carbon and from 1 to 4 heteroatoms selected from the group consisting of O, N and S. In another embodiment, the term heteroaryl refers to a monocyclic, bicyclic or tricyclic aromatic ring of 5- to 14-ring atoms of carbon and from one to four heteroatoms selected from O, N, or S. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. Additional examples of heteroaryl include, but are not limited to pyridyl, e.g., 2-pyridyl (also referred to as α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like. Heterocyclic aromatic (or heteroaryl) as defined above may be optionally substituted with a designated number of substituents, as described below for aromatic groups.

In an embodiment, “heteroaryl” may also include a “fused polycyclic aromatic”, which is a heteroaryl fused with one or more other heteroaryl or nonaromatic heterocyclic ring. Examples include, quinolinyl and isoquinolinyl, e.g. 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g. 2-benzofuranyl and 3-benzofuranyl; dibenzofuranyl, e.g. 2,3-dihydrobenzofuranyl; dibenzothiophenyl; benzothienyl, e.g. 2-benzothienyl and 3-benzothienyl; indolyl, e.g. 2-indolyl and 3-indolyl; benzothiazolyl, e.g., 2-benzothiazolyl; benzooxazolyl, e.g., 2-benzooxazolyl; benzimidazolyl, e.g. 2-benzoimidazolyl; isoindolyl, e.g. 1-isoindolyl and 3-isoindolyl; benzotriazolyl; purinyl; thianaphthenyl, pyrazinyland the like. Fused polycyclic aromatic ring systems may optionally be substituted with a designated number of substituents, as described herein.

The term “heterocycle” or “heterocyclyl” as used herein is intended to mean monocyclic, spirocyclic, bicyclic or tricyclic ring of up to 7 atoms in each ring, wherein each ring is aromatic or non-aromatic and contains carbon and from 1 to 4 heteroatoms selected from the group consisting of O, N, P and S. A nonaromatic heterocycle may be fused with an aromatic aryl group such as phenyl or aromatic heterocycle.

“Heterocyclyl” therefore includes the above mentioned heteroaryls, as well as dihydro and tetrahydro analogs thereof. Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

In an embodiment, “heterocycle” (also referred to herein as “heterocyclyl”), is a monocyclic, spirocyclic, bicyclic or tricyclic saturated or unsaturated ring of 5- to 14-ring atoms of carbon and from one to four heteroatoms selected from O, N, S or P. Examples of heterocyclic rings include, but are not limited to: pyrrolidinyl, piperidinyl, morpholinyl, thiamorpholinyl, piperazinyl, dihydrofuranyl, tetrahydrofuranyl, dihydropyranyl, tetrahydrodropyranyl, dihydroquinolinyl, tetrahydroquinolinyl, dihydroisoquinolinyl, tetrahydroisoquinolinyl, dihydropyrazinyl, tetrahydropyrazinyl, dihydropyridyl, tetrahydropyridyl and the like.

An “alkylaryl group” (arylalkyl) is an alkyl group substituted with an aromatic group, for example, a phenyl group. Another example of an alkylaryl group is a benzyl group. Suitable aromatic groups are described herein and suitable alkyl groups are described herein. Suitable substituents for an alkylaryl group are described herein.

An “alkyheterocyclyl” group” is an alkyl group substituted with a heterocyclyl group. Suitable heterocyclyl groups are described herein and suitable alkyl groups are described herein. Suitable substituents for an alkyheterocyclyl group are described herein.

An “alkycycloalkyl group” is an alkyl group substituted with a cycloalkyl group. Suitable cycloalkyl groups are described herein and suitable alkyl groups are described herein. Suitable substituents for an alkycycloalkyl group are described herein.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

An “alkylamino group” as used herein, is an alkyl group that is attached to a compound via a nitrogen.

As used herein, an “arylalkylamino group” is an arylalkyl group that is attached to a compound via a nitrogen on the alkyl portion of the arylalkyl.

An “alkylsulfonyl group” as used herein, is an alkyl group that is attached to a compound via the sulfur of a sulfonyl group.

As used herein, many moieties or groups are referred to as being either “substituted or unsubstituted”. When a moiety is referred to as substituted, it denotes that any portion of the moiety that is known to one skilled in the art as being available for substitution can be substituted. The phrase “optionally substituted with one or more substituents” means, in one embodiment, one substituent, two substituents, three substituents, four substituents or five substituents. For example, the substitutable group can be a hydrogen atom that is replaced with a group other than hydrogen (i.e., a substituent group). Multiple substituent groups can be present. When multiple substituents are present, the substituents can be the same or different and substitution can be at any of the substitutable sites. Such means for substitution are well known in the art. For purposes of exemplification, which should not be construed as limiting the scope of this invention, some examples of groups that are substituents are: alkyl, alkenyl or alkynyl groups (which can also be substituted, with one or more substituents), alkoxy groups (which can be substituted), a halogen or halo group (F, Cl, Br, I), hydroxy, nitro, oxo, —CN, —COH, —COOH, amino, azido, N-alkylamino or N,N-dialkylamino (in which the alkyl groups can also be substituted), N-arylamino or N,N-diarylamino (in which the aryl groups can also be substituted), esters (—C(O)—OR, where R can be a group such as alkyl, aryl, etc., which can be substituted), ureas (—NHC(O)—NHR, where R can be a group such as alkyl, aryl, etc., which can be substituted), carbamates (—NHC(O)—OR, where R can be a group such as alkyl, aryl, etc., which can be substituted), sulfonamides (—NHS(O)₂R, where R can be a group such as alkyl, aryl, etc., which can be substituted), alkylsulfonyl (which can be substituted), aryl (which can be substituted), cycloalkyl (which can be substituted) alkylaryl (which can be substituted), alkylheterocyclyl (which can be substituted), alkylcycloalkyl (which can be substituted), and aryloxy (which can be substituted).

In one embodiment, A is phenyl, thienyl or pyridyl, optionally substituted with halo, methyl, methoxy amino, hydroxyl or halomethyl. In one embodiment, A is

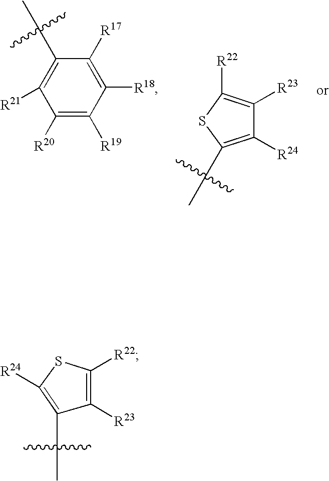

R¹⁷ and R²¹ are independently selected from hydrogen or fluoro;

R¹⁸, R¹⁹ or R²⁰ are independently selected from hydrogen, halo, methyl, methoxy or halomethyl;

R²², R²³ and R²⁴ are independently selected from hydrogen, methyl, amino, hydroxyl, and halo.

In another embodiment, A is H.

In one embodiment, A is

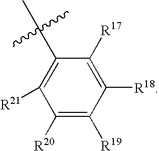

In one embodiment, R¹⁷ and R²¹ are independently selected from hydrogen or fluoro; R¹⁸, R¹⁹ or R²⁰ are independently selected from hydrogen, halo, methyl, methoxy or halomethyl.

In another embodiment, R¹⁷, R¹⁸, R²⁰, and R²¹ are independently selected from hydrogen or fluoro; R¹⁹ is independently selected from hydrogen, halo, methyl, methoxy or halomethyl.

In one embodiment, A is

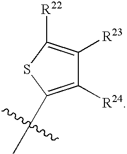

In another embodiment, A is

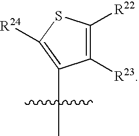

In one embodiment, R²², R²³ and R²⁴ are independently selected from hydrogen, methyl, and halo.

In another embodiment, A is phenyl or thienyl. In a further embodiment, A is phenyl.

In one embodiment, R¹ and R² are independently selected from H, OH, halo, NH₂, C₁-C₄ alkyl, or C₁-C₁₀ alkoxy. In one embodiment, R¹ and R² are independently selected from H, OH, halo, NH₂, C₁-C₂ alkyl, or C₁-C₂ alkoxy. In another embodiment, R¹ and R² are H. In one embodiment, R¹ and R² are independently selected from H, OH, halo, NH₂, C₁-C₄ alkyl, C₁-C₄ alkenyl, C₁-C₄ alkynyl, C₁-C₄ alkoxy, C₃-C₆ cycloalkyl, heteroaryl, heterocyclic or aryl, wherein the cycloalkyl, heteroaryl, heterocyclic or aryl is optionally substituted with OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino.

In one embodiment, R³ is H.

In another embodiment, R⁴ is —NR⁶R⁷.

In one embodiment, R⁵ is H. In another embodiment, R⁵ is independently selected from H, OH, NH₂, nitro, CN, amide, carboxyl, C₁-C₂ alkoxy, C₁-C₂ alkyl, C₁-C₂ haloalkyl, C₁-C₂ haloalkyloxy, C₁-C₂ hydroxyalkyl, C₁-C₂ alkenyl, C₁-C₂ alkyl-C(═O)O—, C₁-C₂ alkyl-C(═O)—, C₁-C₂ alkynyl, halo, hydroxyalkoxy, C₁-C₂ alkyl-NHSO₂—, C₁-C₂ alkyl-SO₂NH—, C₁-C₂ alkylsulfonyl, C₁-C₂ alkylamino or di(C₁-C₂)alkylamino. In a further embodiment, R⁵ is independently selected from H, OH, NH₂, nitro, CN, amide, carboxyl, C₁-C₄ alkoxy, C₁-C₄ alkyl, C₁-C₄ haloalkyl, C₁-C₄ haloalkyloxy, C₁-C₄ hydroxyalkyl, C₁-C₄ alkenyl, C₁-C₄ alkyl-C(═O)O—, C₁-C₄ alkyl-C(═O)—, C₁-C₄ alkynyl, halo, hydroxyalkoxy, C₁-C₄ alkyl-NHSO₂—, C₁-C₄ alkyl-SO₂NH—, C₁-C₄ alkylsulfonyl, C₁-C₄ alkylamino or di(C₁-C₄)alkylamino.

In one embodiment, R⁶ is selected from H or C₁-C₄ alkyl. In one embodiment, R⁶ is selected from H or C₁-C₂ alkyl. In one embodiment, R⁶ is H.

In another embodiment, R⁷ is —(O)(CR^{a}₂)_{q}R¹². In one embodiment, R⁷ is —C(O)O(CR^{a}₂)_{q}R¹². In one embodiment, R⁷ is —C(O)OCH₂R¹².

In one embodiment, R¹² is selected from C₁-C₄ alkyl, C₃-C₆ cycloalkyl, heteroaryl, aryl or heterocyclic, wherein the alkyl, cycloalkyl, heteroaryl, heterocyclic or aryl is attached to a fluorophore through a linker, and optionally substituted with aryl, heteroaryl, halo, C₁-C₄ alkyl, N(R⁶)₂, OH, C₁-C₄ alkoxy or C₁-C₄ haloalkyl. In one embodiment, R¹² is selected from C₁-C₄ alkyl, C₃-C₆ cycloalkyl, heteroaryl, aryl or heterocyclic, wherein the alkyl, cycloalkyl, heteroaryl, heterocyclic or aryl is attached to a fluorophore through a linker and optionally substituted with OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—C₁-C₇ alkylsulfonyl, C₁-C₇ alkylamino or di(C₁-C₇)alkylamino, aryl, heterocyclic or cycloalkyl.

In one embodiment, R¹² is selected from heterocyclic, heteroaryl or aryl, attached to a fluorophore through a linker. In another embodiment, R¹² is selected from heteroaryl or aryl, attached to a fluorophore through a linker. In a further embodiment, R¹² is selected from phenyl or 2-pyridyl attached to a fluorophore through a linker. In a further embodiment, R¹² is furanyl, thiophenyl or pyranyl attached to a fluorophore through a linker. In one embodiment, R¹² is phenyl attached to a fluorophore through a linker.

In one embodiment, R¹² is optionally substituted with aryl, heteroaryl, halo, C₁-C₄ alkyl, N(R⁶)₂, OH, C₁-C₄ alkoxy or C₁-C₄ haloalkyl. In one embodiment, R¹² is optionally substituted with OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino, aryl, heterocyclic or cycloalkyl.

In another embodiment, R¹² is optionally substituted with OH, NH₂, nitro, CN, amide, carboxyl, C₁-C₄ alkoxy, C₁-C₄ alkyl, C₁-C₄ haloalkyl, C₁-C₄ haloalkyloxy, C₁-C₄ hydroxyalkyl, C₁-C₄ alkenyl, C₁-C₄ alkyl-C(═O)O—, C₁-C₄ alkyl-C(═O)—, C₁-C₄ alkynyl, halo, hydroxyalkoxy, C₁-C₄ alkyl-NHSO₂—, C₁-C₄ alkyl-SO₂NH—, C₁-C₄ alkylsulfonyl, C₁-C₄ alkylamino or di(C₁-C₄)alkylamino.

In a further embodiment, R¹² is optionally substituted with OH, NH₂, nitro, CN, amide, carboxyl, C₁-C₂ alkoxy, C₁-C₂ alkyl, C₁-C₂ haloalkyl, C₁-C₂ haloalkyloxy, C₁-C₂ hydroxyalkyl, C₁-C₂ alkenyl, C₁-C₂ alkyl-C(═O)O—, C₁-C₂ alkyl-C(═O)—, C₁-C₂ alkynyl, halo, hydroxyalkoxy, C₁-C₂ alkyl-NHSO₂—, C₁-C₂ alkyl-SO₂NH—, C₁-C₂ alkylsulfonyl, C₁-C₂ alkylamino or di(C₁-C₂)alkylamino.

In a further embodiment, R¹² is optionally substituted with C₁-C₂ alkyl. In a further embodiment, R¹² is optionally substituted with C₁-C₄ alkyl.

In one embodiment, R^{a} is H. In another embodiment, R^{a} is H or C₁-C₂ alkyl. In another embodiment, R^{a} is H or C₁-C₄ alkyl.

In one embodiment, Ring B is selected from phenyl, benzothiophenyl, benzofuranyl, thiazolyl, benzothiazolyl, furanyl, pyridyl, pyrimidyl, quinolinyl, thiophenyl, benzodioxyl, benzooxadiazolyl, quinoxalinyl, benzotriazolyl, benzoimidazolyl or benzooxazolyl. In another embodiment, Ring B is phenyl, benzothiophenyl, thiophenyl or pyridyl. In a further embodiment, Ring B is phenyl or pyridyl. In a further embodiment, Ring B is phenyl.

In one embodiment, n is 1 or 2. In another embodiment, n is 1.

In one embodiment, p is 1, 2, 3 or 4. In another embodiment, p is 1.

In one embodiment, q is independently 0, 1, 2, 3, or 4. In another embodiment, q is independently 0, 1 or 2. In a further embodiment, q is 0. In a further embodiment, q is 1. In a further embodiment, q is 2.

In one embodiment, L¹ is ethenyl or a bond. In another embodiment, L¹ is a bond.

In one embodiment, X is OH or NH₂. In another embodiment, X is NIH2.

In another embodiment, Z is C.

In one embodiment, R²⁶ to R²⁹ is independently selected from H, C₁-C₄ alkyl, CN, azido, C₁-C₄ cyanoalkyl, nitro, halo, C₁-C₄ haloalkyl, amino, C₁-C₄ alkoxycarbonyl, C₁-C₄ alkylaminocarbonyl, hydroxyl, C₁-C₄ alkoxy or aryl-C₁-C₄-alkoxy.

In one embodiment, R³⁰ is H.

In one embodiment, R³¹ is H.

In another embodiment, n is 6.

In one embodiment, m is 0 or 1. In one embodiment, m is 1.

In one embodiment, the linker is

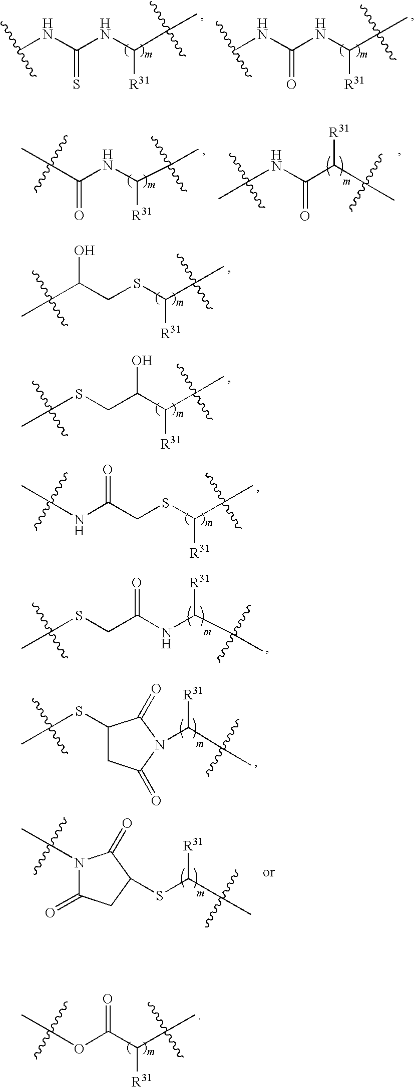

In one embodiment, the linker is

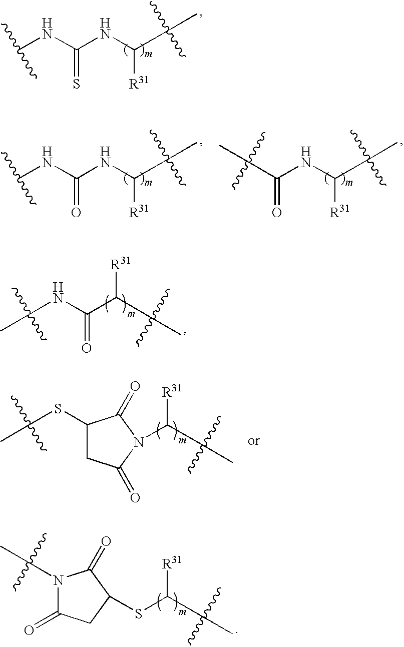

In one embodiment, the linker is

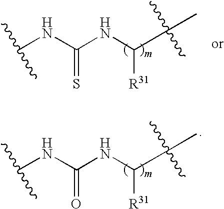

In another embodiment, the linker is

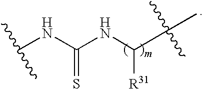

### Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the Formulas of the invention, it is understood that both the (R) and (S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the HDAC inhibitors of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

### Pharmaceutically Acceptable Salts

The fluorescent compounds described herein can, as noted above, can be prepared in the form of their pharmaceutically acceptable salts. Pharmaceutically acceptable salts are salts that retain the desired biological activity of the parent compound and do not impart undesired toxicological effects. Examples of such salts are (a) acid addition salts organic and inorganic acids, for example, acid addition salts which may, for example, be hydrochloric acid, sulphuric acid, methanesulphonic acid, fumaric acid, maleic acid, succinic acid, acetic acid, benzoic acid, oxalic acid, citric acid, tartaric acid, carbonic acid, phosphoric acid, trifluoroacetic acid, formic acid and the like. Pharmaceutically acceptable salts can also be prepared from by treatment with inorganic bases, for example, sodium, potassium, ammonium, calcium, or ferric hydroxides, and such organic bases as isopropylamine, trimethylamine, 2-ethylamino ethanol, histidine, procaine, and the like. Pharmaceutically acceptable salts can also be formed from elemental anions such as chlorine, bromine and iodine.

The compounds disclosed can, as noted above, also be prepared in the form of their hydrates. The term “hydrate” includes but is not limited to hemihydrate, monohydrate, dihydrate, trihydrate, tetrahydrate and the like.

The compounds disclosed can, as noted above, also be prepared in the form of a solvate with any organic or inorganic solvent, for example alcohols such as methanol, ethanol, propanol and isopropanol, ketones such as acetone, aromatic solvents and the like.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

### Methods of Treatment

As demonstrated herein, the HDAC inhibitors are useful for the treatment of cancer. In addition, there is a wide range of other diseases for which the HDAC inhibitors may be found useful. Non-limiting examples are thioredoxin (TRX)-mediated diseases as described herein, and diseases of the central nervous system (CNS) as described herein.

### 1. Treatment of Cancer

As demonstrated herein, the HDAC inhibitors are useful for the treatment of cancer. The term “cancer” refers to any cancer caused by the proliferation of neoplastic cells, such as solid tumors, neoplasms, carcinomas, sarcomas, leukemias, lymphomas and the like. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

### 2. Treatment of Thioredoxin (TRX)-Mediated Diseases

Examples of TRX-mediated diseases include, but are not limited to, acute and chronic inflammatory diseases, autoimmune diseases, allergic diseases, diseases associated with oxidative stress, and diseases characterized by cellular hyperproliferation.

Non-limiting examples are inflammatory conditions of a joint including rheumatoid arthritis (RA) and psoriatic arthritis; inflammatory bowel diseases such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fascitis; cancers with leukocyte infiltration of the skin or organs, ischemic injury, including cerebral ischemia (e.g., brain injury as a result of trauma, epilepsy, hemorrhage or stroke, each of which may lead to neurodegeneration); HIV, heart failure, chronic, acute or malignant liver disease, autoimmune thyroiditis; systemic lupus erythematosus, Sjorgren's syndrome, lung diseases (e.g., ARDS); acute pancreatitis; amyotrophic lateral sclerosis (ALS); Alzheimer's disease; cachexia/anorexia; asthma; atherosclerosis; chronic fatigue syndrome, fever; diabetes (e.g., insulin diabetes or juvenile onset diabetes); glomerulonephritis; graft versus host rejection (e.g., in transplantation); hemohorragic shock; hyperalgesia: inflammatory bowel disease; multiple sclerosis; myopathies (e.g., muscle protein metabolism, esp. in sepsis); osteoporosis; Parkinson's disease; pain; pre-term labor; psoriasis; reperfusion injury; cytokine-induced toxicity (e.g., septic shock, endotoxic shock); side effects from radiation therapy, temporal mandibular joint disease, tumor metastasis; or an inflammatory condition resulting from strain, sprain, cartilage damage, trauma such as burn, orthopedic surgery, infection or other disease processes. Allergic diseases and conditions, include but are not limited to respiratory allergic diseases such as asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies, and the like.

### 3. Treatment of Diseases of the Central Nervous System (CNS)

CNS disease includes neurodegenerative disease, inherited neurodegenerative disease, such as those inherited neurodegenerative diseases that are polyglutamine expansion diseases. Generally, neurodegenerative diseases can be grouped as follows:

I. Disorders characterized by progressive dementia in the absence of other prominent neurologic signs, such as Alzheimer's disease; Senile dementia of the Alzheimer type; and Pick's disease (lobar atrophy).
II. Syndromes combining progressive dementia with other prominent neurologic abnormalities such as A) syndromes appearing mainly in adults (e.g., Huntington's disease, Multiple system atrophy combining dementia with ataxia and/or manifestations of Parkinson's disease, Progressive supranuclear palsy (Steel-Richardson-Olszewski), diffuse Lewy body disease, and corticodentatonigral degeneration); and B) syndromes appearing mainly in children or young adults (e.g., Hallervorden-Spatz disease and progressive familial myoclonic epilepsy).
III. Syndromes of gradually developing abnormalities of posture and movement such as paralysis agitans (Parkinson's disease), striatonigral degeneration, progressive supranuclear palsy, torsion dystonia (torsion spasm; dystonia musculorum deformans), spasmodic torticollis and other dyskinesis, familial tremor, and Gilles de la Tourette syndrome.
IV. Syndromes of progressive ataxia such as cerebellar degenerations (e.g., cerebellar cortical degeneration and olivopontocerebellar atrophy (OPCA)); and spinocerebellar degeneration (Friedreich's atazia and related disorders).
V. Syndrome of central autonomic nervous system failure (Shy-Drager syndrome).
VI. Syndromes of muscular weakness and wasting without sensory changes (motomeuron disease such as amyotrophic lateral sclerosis, spinal muscular atrophy (e.g., infantile spinal muscular atrophy (Werdnig-Hoffman), juvenile spinal muscular atrophy (Wohlfart-Kugelberg-Welander) and other forms of familial spinal muscular atrophy), primary lateral sclerosis, and hereditary spastic paraplegia.
VII. Syndromes combining muscular weakness and wasting with sensory changes (progressive neural muscular atrophy; chronic familial polyneuropathies) such as peroneal muscular atrophy (Charcot-Marie-Tooth), hypertrophic interstitial polyneuropathy (Dejerine-Sottas), and miscellaneous forms of chronic progressive neuropathy.
VIII. Syndromes of progressive visual loss such as pigmentary degeneration of the retina (retinitis pigmentosa), and hereditary optic atrophy (Leber's disease).

### Histone Deacetylases and Histone Deacetylase Inhibitors

Histone deacetylases (HDACs), as that term is used herein, are enzymes that catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation and/or apoptosis of transformed cells in vitro and inhibit tumor growth in vivo. HDACs can be divided into three classes based on structural homology. Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors, as that term is used herein are compounds that are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures that can assay for the accumulation of acetylated histones can be used to determine the HDAC inhibitory activity of compounds of interest. It is understood that compounds that can inhibit histone deacetylase activity can also bind to other substrates and as such can inhibit other biologically active molecules such as enzymes. It is also to be understood that the compounds of the present invention are capable of inhibiting any of the histone deacetylases set forth above, or any other histone deacetylases.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assays which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92:1210-1215, 2000, Butler, L. M. et al., Cancer Res. 60:5165-5170 (2000), Richon, V. M. et al., Proc. Natl. Acad. Sci., USA, 95:3003-3007, 1998, and Yoshida, M. et al., J. Biol. Chem., 265:17174-17179, 1990.

For example, an enzymatic assay to determine the activity of an HDAC inhibitor compound can be conducted as follows. Briefly, the effect of an HDAC inhibitor compound on affinity purified human epitope-tagged (Flag) HDAC1 can be assayed by incubating the enzyme preparation in the absence of substrate on ice for about 20 minutes with the indicated amount of inhibitor compound. Substrate ([³H]acetyl-labelled murine erythroleukemia cell-derived histone) can be added and the sample can be incubated for 20 minutes at 37° C. in a total volume of 30 μL. The reaction can then be stopped and released acetate can be extracted and the amount of radioactivity release determined by scintillation counting. An alternative assay useful for determining the activity of an HDAC inhibitor compound is the “HDAC Fluorescent Activity Assay; Drug Discovery Kit-AK-500” available from BIOMOL Research Laboratories, Inc., Plymouth Meeting, Pa.

In vivo studies can be conducted as follows. Animals, for example, mice, can be injected intraperitoneally with an HDAC inhibitor compound. Selected tissues, for example, brain, spleen, liver etc, can be isolated at predetermined times, post administration. Histones can be isolated from tissues essentially as described by Yoshida et al., J. Biol. Chem. 265:17174-17179, 1990. Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

In addition, hydroxamic acid-based HDAC inhibitors have been shown to up regulate the expression of the p21^{WAF1} gene. The p21^{WAF1} protein is induced within 2 hours of culture with HDAC inhibitors in a variety of transformed cells using standard methods. The induction of the p21^{WAF1} gene is associated with accumulation of acetylated histones in the chromatin region of this gene. Induction of p21^{WAF1} can therefore be recognized as involved in the G1 cell cycle arrest caused by HDAC inhibitors in transformed cells.

The invention is illustrated in the examples in the Experimental Details Section that follows. This section is set forth to aid in an understanding of the invention but is not intended to, and should not be construed to limit in any way the invention as set forth in the claims which follow thereafter.

### EXPERIMENTAL DETAILS SECTION

### Example 1

### Synthesis

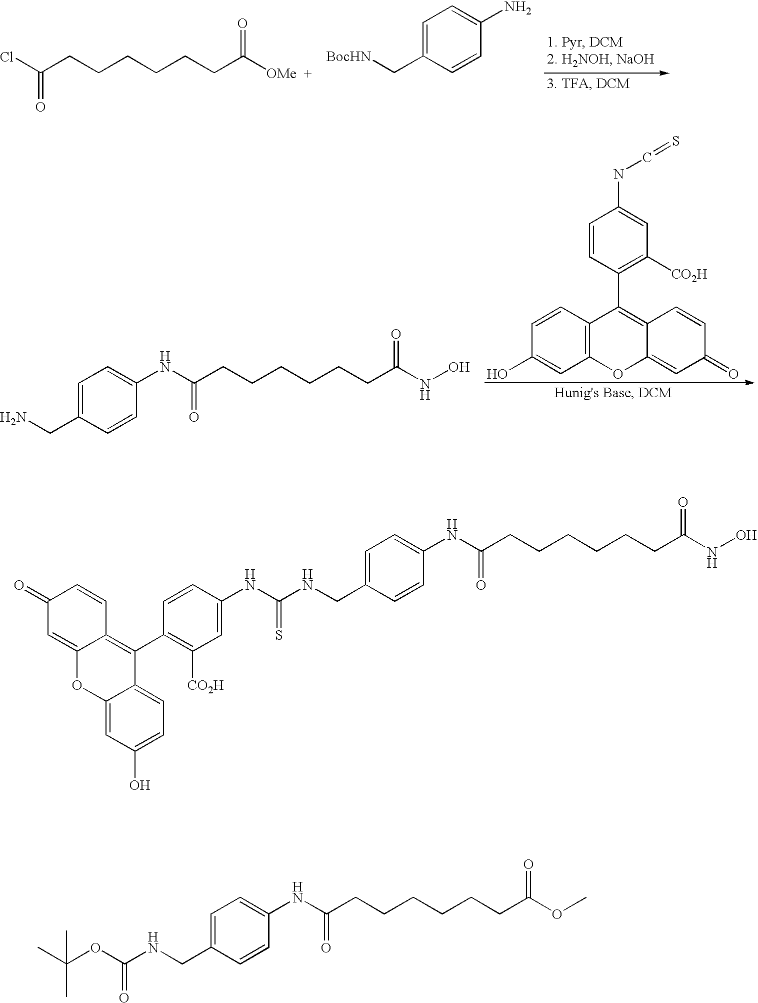

Methyl-8-[(4-{[(tert-butoxycarbonyl)amino]methyl}phenyl)amino]-8-oxooctanoate. tert-Butyl (4-aminobenzyl)carbamate (3.0 g, 13.5 mmol) was made 0.25 M in anhydrous DCM and to this stirring solution was added Pyridine (1.6 g, 20.2 mmol) followed by methyl 8-chloro-8-oxooctanoate (2.8 g, 13.5 mmol). The resulting solution was stirred at ambient temperature and reaction progress was monitored by LC/MS. The reaction mixture was stirred for 16 hours then diluted with ethyl acetate and washed with aq 1N HCl. The organic layer was again washed with aqueous 1N HCl, brine then dried over anhydrous MgSO₄ and concentrated in vacuo to give the title compound as a white solid. cal'd [M+H]⁺ 393, exp. 393

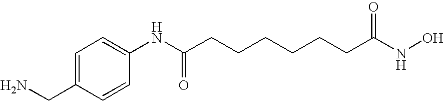

N-[4-(aminomethyl)phenyl]-N′-hydroxyoctanediamide. Methyl-8-[(4-{[(tert-butoxycarbonyl)amino]methyl}phenyl)amino]-8-oxooctanoate (2.0 g, 5.1 mmol)) was made 0.25 M in MeOH and to this stirring solution was added hydroxylamine (0.2 g, 6.1 mmol) followed by 5N potassium hydroxide (7.1 mL, 35.7 mmol). The resulting solution was stirred at ambient temperature for 16 hours. The reaction mixture was then acidified with TFA and concentrated in vacuo. The residue was stirred in 2:1 DCM:TFA for 3 hours. The reaction mixture was concentrated in vacuo, dissolved in MeOH, and purified by HPLC (2-65% ACN:H2O with 0.025% TFA). Pure fractions were identified, combined, and concentrated in vacuo to give the title compound as a TFA salt. cal'd [M+H]⁺ 294, exp. 294

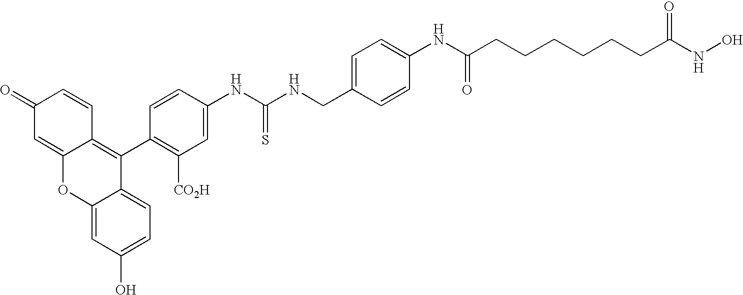

5-({[(4-{[8-(hydroxyamino)-8-oxooctanoyl]amino}benzyl)amino]carbonothioyl}amino)-2-(6-hydroxy-3-oxo-3H-xanthen-9-yl)benzoic acid (COMPOUND 1). N-[4-(aminomethyl)phenyl]-N′-hydroxyoctanediamide (345 mg, 1.18 mmol)) was made 0.1 M in anhydrous 1:1 DMF:DCM and to this stirring solution was added DIPEA (456 mg, 3.53 mmol) followed by fluoroscein-5-isothiocyanate (229 mg, 0.59 mmol). The resulting solution was stirred at ambient temperature for 3 hours then the mixture was purified by HPLC (Solvent A=0.1M TEA:H₂O adjusted to pH7 with AcOH, Solvent B=9:1 ACN:H₂O; Gradient=0% B for 2 mins, ramp to 30% B over 12 mins, then ramp to 95% B over 15 mins). Pure fractions were identified and lyophilized to give the title compound. ¹H NMR (CDOD, 600 MHz) [symbol] 8.04 (d, J=1.8 Hz, 1H), 7.73 (dd, J=8.2, 2.1 Hz, 1H), 7.52 (d, J=8.5 Hz, 2H), 7.34 (d, J=8.5 Hz, 2H), 7.17 (d, J=8.2 Hz, 1H), 6.95 (d, J=9.1 Hz, 2H), 6.65 (d, J=2.4 Hz, 2H), 6.58 (dd, J=9.1, 2.3 Hz, 2H), 4.82 (s, 2H), 2.35 (t, J=7.3 Hz, 2H), 2.08 (t, J=7.3 Hz, 2H), 1.58-1.72 (m, 4H), 1.32-1.44 (m, 4H). cal'd [M+H]⁺ 683, exp. 683

Other methods of synthesizing hydroxamic acid HDAC inhibitors are disclosed in U.S. Pat. Nos. 5,369,108, 5,932,616, 5,700,811, 6,087,367 and 6,511,990, these synthesis methods are incorporated herein by reference.

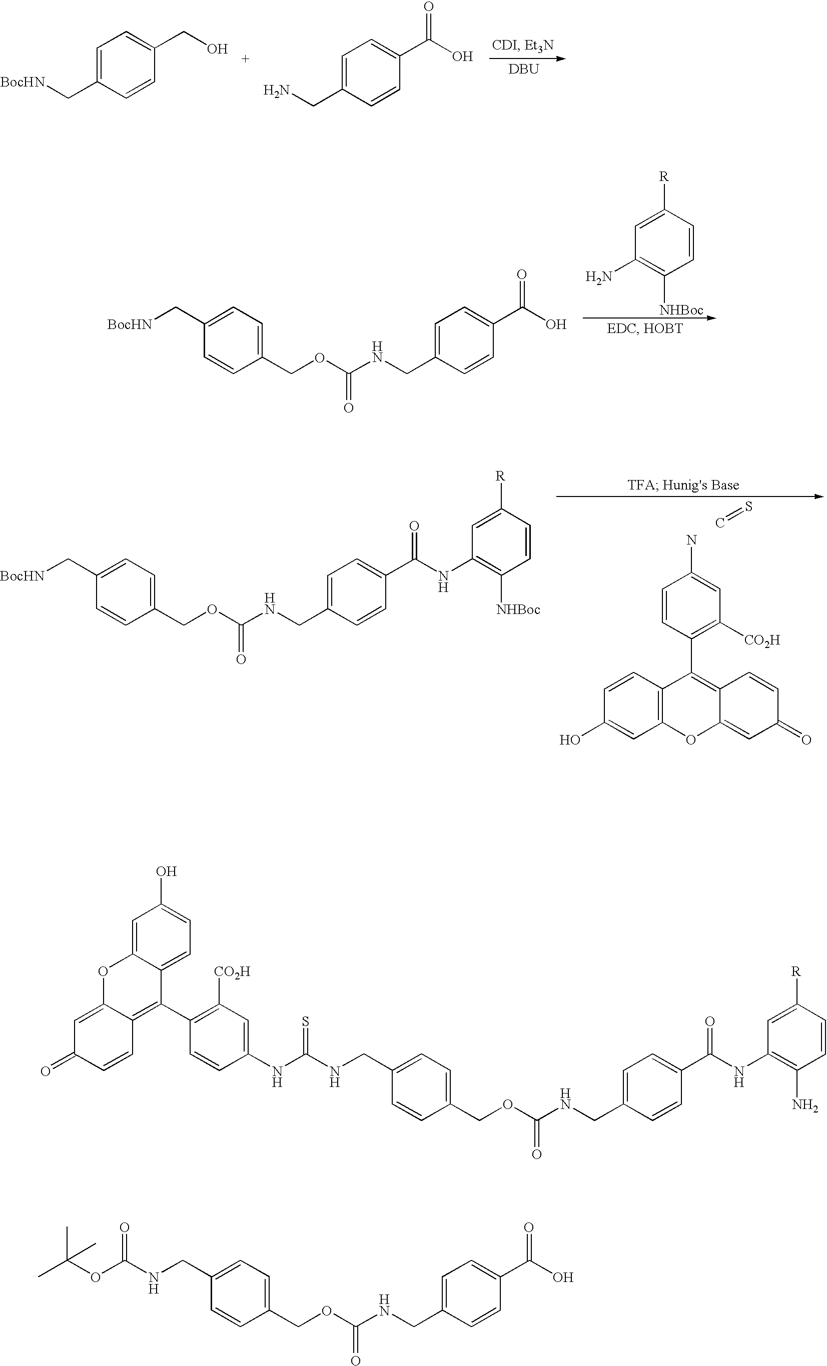

4-[({[(4-{[(tert-butoxycarbonyl)amino methyl}benzyl)oxy]carbonyl}amino) methyl]benzoic acid. To a suspension of CDI (340 mgd, 2.1 mmol) in THF (1.6 mL) was added tert-butyl [4-(hydroxymethyl)benzyl]carbamate (500 mg, 2.1 mmol)) in THF (0.7 mL) and the mixture was aged for 1 hour at ambient temperature. The resulting mixture was then added to a stirring solution of 4-(aminomethyl)benzoic acid (320 mg, 2.1 mmol), TEA (0.3 mL, 2.1 mmol), and DBU (0.3 mL, 2.1 mmol)) in THF (3.5 mL). After stirring at ambient temperature for 5 hours the reaction mixture was concentrated in vacuo, diluted with water, then acidified with HCl. The white precipitate was filtered away, washed with water then dissolved in DCM:EtOAc (some MeOH was added for solubility) dried over anhydrous MgSO₄ and concentrated in vacuo to give the title compound. cal'd [M+Na]+437, exp. 437

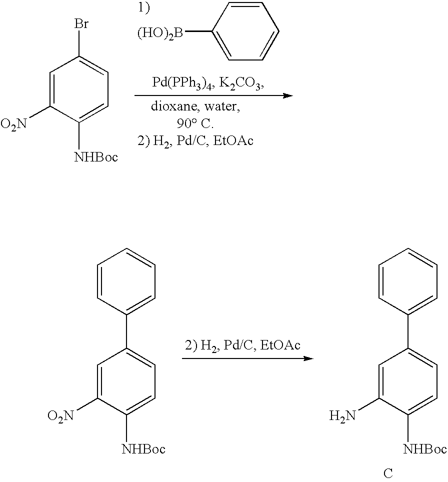

tert-Butyl (3-Aminobiphenyl-4-yl)carbamate (C). Intermediate C was prepared from tert-butyl (4-bromo-2-nitrophenyl)carbamate as described in a published procedure; see Adam, G.; Alanine, A.; Goetschi, E.; Mutel, V.; Woltering, T. J. Preparation of benzodiazepine derivatives as metabotropic glutamate receptor antagonists. PCT Int. Appl. (2001) WO 2001029012 A2.

A mixture of N-Boc 4-bromo-2-nitroaniline (39.0 g, 123 mmol), phenylboronic acid (16.5 g, 135 mmol) and K₂CO₃ (34.1 g, 247 mmol) in 350 mL of dioxane and 150 mL of water was degassed by bubbling nitrogen through the mixture for 30 min. Next, Pd(PPh₃)₄ was added (4.32 g, 3.7 mmol) and the orange mixture was warmed to 78° C. for 18 h. Cooled and partitioned between ether (1500 mL) and water (400 mL). Filtered mixture through a pad of Celite (w/ether washes). Organic layer was separated, washed with brine, dried (MgSO₄) and concentrated to afford 44.1 g of reddish-orange solid. Recrystallization from EtOAc-hexanes (ca. 50 mL+1100 mL, respectively) afforded the bright orange solid N-Boc 4-phenyl-2-nitroaniline: MS (EI) [M+Na]⁺ cal'd 337.2, obs'd 337.2.

A solution of nitro compound (16.5 g, 52.5 mmol) in 400 mL of EtOAc evacuated and refilled with nitrogen (2×). Added 10% Pd/C (1.60 g), then evacuated and refilled with hydrogen (3×). Stirred under atmosphere of hydrogen overnight. Mixture was filtered through a pad of Celite (w/EtOAc, then CH₂Cl₂ washes) and concentrated to a pale orange solid. Stirred and warmed with ca. 800 mL of hexanes, then cooled and collected product (w/cold hexane washes). Dissolved resulting solid in CH₂Cl₂ and concentrated to provide the off-white solid N—BOC (3-aminobiphenyl-4-yl)amine C: ¹H NMR (600 MHz, CDCl₃) δ 7.51 (d, J=3.2 Hz, 2H), 7.38 (t, J=5.6 Hz, 2H), 7.31 (m, 2H), 7.22 (s, 1H), 7.12 (dd, J=8.2, 2.1 Hz, 1H), 6.45 (br s, 1H), 1.51 (s, 9H); MS (EI) [M+Na]⁺ cal'd 285.1, obs'd 285.1.

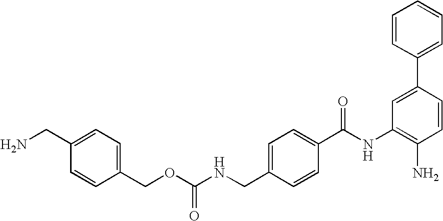

4-(aminomethyl)benzyl(4-{[(4-aminobiphenyl-3-yl)amino]carbonyl}benzyl)carbamate. 4-[({[(4-{[(tert-butoxycarbonyl)amino]methyl}benzyl)oxy]carbonyl}amino) methyl]benzoic acid (0.40 g, 0.97 mmol), tert-butyl (3-aminobiphenyl-4-yl)carbamate (0.30 g, 1.06 mmol), EDCI (0.22 g, 1.16 mmol), and HOBT (0.18 g, 1.16 mmol) were stirred in DMF (3.9 mL) at ambient temperature for 48 hours. The reaction mixture was diluted with water and extracted with EtOAc 2×. The combined organic layers was washed with 1 N aq HCl (2×), then washed with saturated aqueous sodium bicarbonate, brine, dried over anhydrous MgSO₄ and concentrated in vacuo. The residue was then diluted with 2:1 DCM:TFA and stirred at ambient temperature for 3 hours. The reaction mixture was then concentrated in vacuo, diluted with MeOH and purified by HPLC (5-75% ACN:H2O with 0.025% TFA). Pure fractions were identified, combined and concentrated in vacuo to give the title compound as a bis TFA salt. cal'd [M+H]⁺ 481, exp. 481

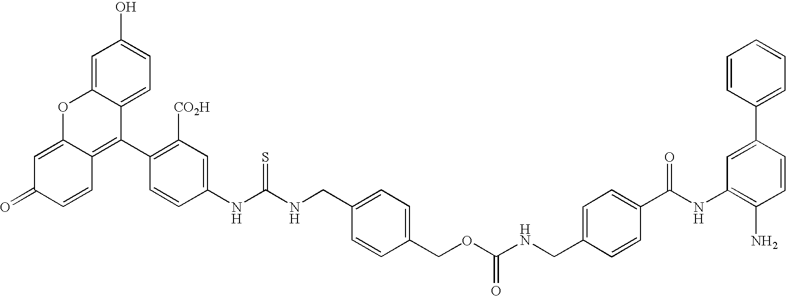

5-{[({4-[({[(4-{[(4-aminobiphenyl-3-yl)amino]carbonyl}benzyl)amino]carbonyl}oxy)methyl]benzyl}amino)carbonothioyl]amino}-2-(6-hydroxy-3-oxo-3H-xanthen-9-yl)benzoic acid (COMPOUND 2). 4-(aminomethyl)benzyl(4-{[(4-aminobiphenyl-3-yl)amino]carbonyl}benzyl)carbamate (200 mg, 0.34 mmol)) was made 0.1 M in anhydrous 1:1 DMF:DCM and to this stirring solution was added DIPEA (130 mg, 1.01 mmol) followed by fluoroscein-5-isothiocyanate (70 mg, 0.17 mmol). The resulting solution was stirred at ambient temperature for 16 hours then the mixture was purified by HPLC (Solvent A=0.1M TEA:H₂O adjusted to pH7 with AcOH, Solvent B=9:1 ACN:H₂O; Gradient=0% B for 2 mins, ramp to 30% B over 12 mins, then ramp to 95% B over 15 mins). Pure fractions were identified and lyophilized to give the title compound as an acetic acid salt. ¹H NMR (DMSO-d₆, 600 MHz) δ11.07 (br s, 1H), 9.70 (br s, 1H), 9.57 (br s, 1H), 8.31 (br s, 1H), 7.88-7.94 (m, 3H), 7.80 (dd, J=8.2, 1.8 Hz, 1H), 7.45-7.55 (m, 3H), 7.25-7.4 (m, 9H), 7.20 (t, J=7.3 Hz, 1H), 7.12 (d, J=8.2 Hz, 1H), 6.83 (d, J=8.5 Hz, 1H), 6.63 (d, J=2.3 Hz, 2H), 6.58 (d, J=8.5 Hz, 2H), 6.52 (dd, J=8.8, 2.3 Hz, 2H), 5.07 (br s, 2H), 5.02 (s, 2H), 4.75 (s, 2H), 4.26 (d, J=6.2 Hz, 2H) 1.87 (s, 3H). cal'd [M+H]⁺ 870, exp. 870

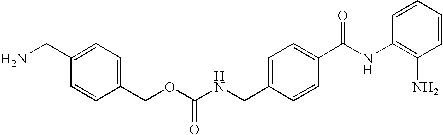

4-(aminomethyl)benzyl(4-{[(2-aminophenyl)amino]carbonyl}benzyl)carbamate. 4-[({[(4-{[(tert-butoxycarbonyl)amino]methyl}benzyl)oxy]carbonyl}amino) methyl]benzoic acid (0.20 g, 0.48 mmol), tert-butyl (2-aminophenyl)carbamate (0.12 g, 0.58 mmol), EDCI (0.11 g, 0.58 mmol), and HOBT (0.89 g, 0.58 mmol) were stirred in DMF (1.9 mL) at ambient temperature for 48 hours. The reaction mixture was diluted with water and extracted with EtOAc (2×). The combined organic layers was washed with 1 N aq HCl (2×), then washed with saturated aqueous sodium bicarbonate, brine, dried over anhydrous MgSO₄ and concentrated in vacuo. The residue was then diluted with 2:1 DCM:TFA and stirred at ambient temperature for 3 hours. The reaction mixture was concentrated in vacuo, diluted with EtOAc and washed with saturated aqueous sodium bicarbonate. An emulsion formed and a fine precipitate came out of solution. The precipitate was filtered away. LC/MS and ¹H-NMR indicated it was pure desired product. The material was carried forward without further purification. cal'd [M+H]⁺ 405, exp. 405.

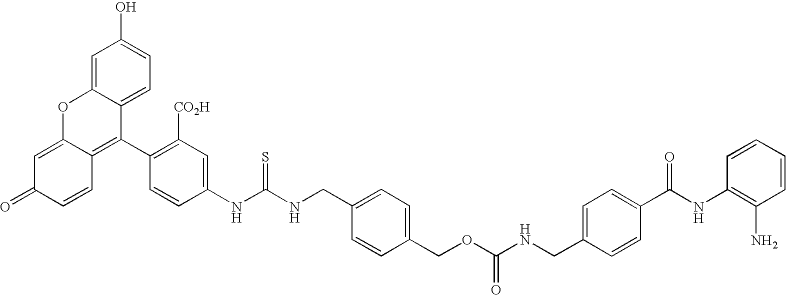

5-{[({4-[({[(4-{[(2-aminophenyl)amino]carbonyl}benzyl)amino]carbonyl}oxy)methyl]benzyl}amino)carbonothioyl]amino}-2-(6-hydroxy-3-oxo-3H-xanthen-9-yl)benzoic acid. (COMPOUND 3) 4-(aminomethyl)benzyl(4-{[(2-aminophenyl)amino]carbonyl}benzyl)carbamate (150 mg, 0.29 mmol)) was made 0.1 M in anhydrous 1:1 DMF:DCM and to this stirring solution was added DIPEA (112 mg, 0.87 mmol) followed by fluoroscein-5-isothiocyanate (56 mg, 0.15 mmol). The resulting solution was stirred at ambient temperature for 16 hours then the mixture was purified by HPLC (Solvent A=0.1 M TEA:H₂O adjusted to pH7 with AcOH, Solvent B=9:1 ACN:H₂O; Gradient=0% B for 2 mins, ramp to 30% B over 12 mins, then ramp to 95% B over 15 mins). Pure fractions were identified and lyophilized to give the title compound as an acetic acid salt. ¹H NMR (DMSO-d₆, 600 MHz) [symbol] 10.12 (br s, 2H), 8.27-8.31 (m, 2H), 7.84-7.94 (m, 4H), 7.62 (br s, 1H), 7.50 (br s, 1H), 7.29-7.35 (m, 5H), 7.17 (br s, 2H), 7.09 (d, J=7.9 Hz, 2H), 6.49-6.59 (m, 11H), 5.01 (s, 2H), 4.74 (s, 2H), 4.23 (s, 2H), 1.87 (s, 3H).

Below are some examples of general methods to synthesize HDAC inhibitors that can be attached to the fluorophore through a linker. In the following schemes, Ar represents aryl and Het represents heteroaryl.

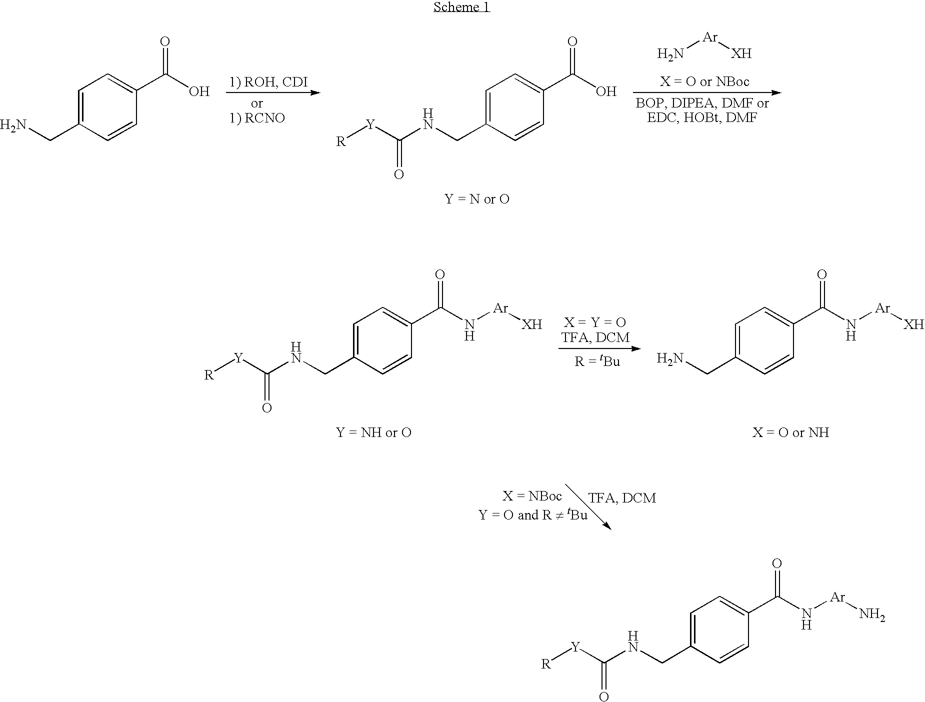

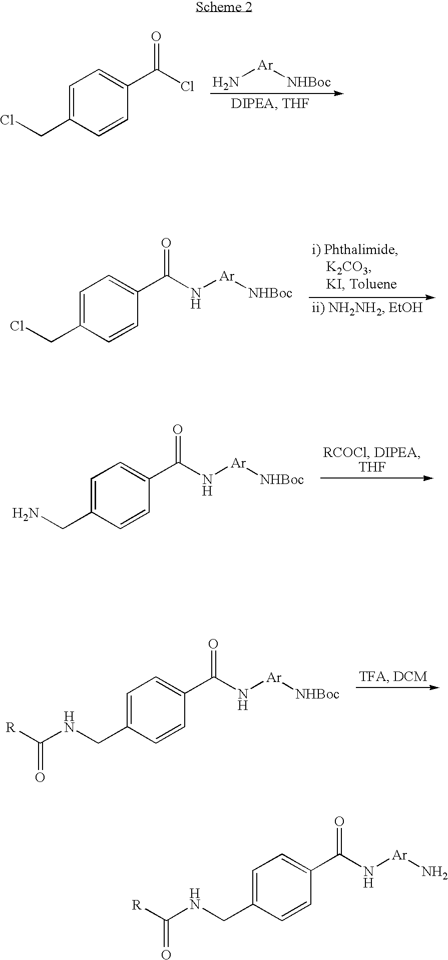

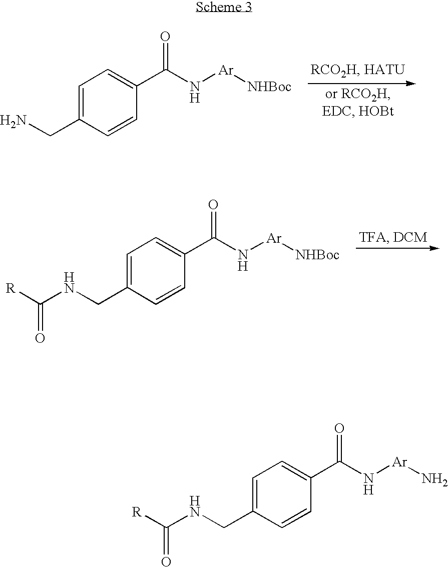

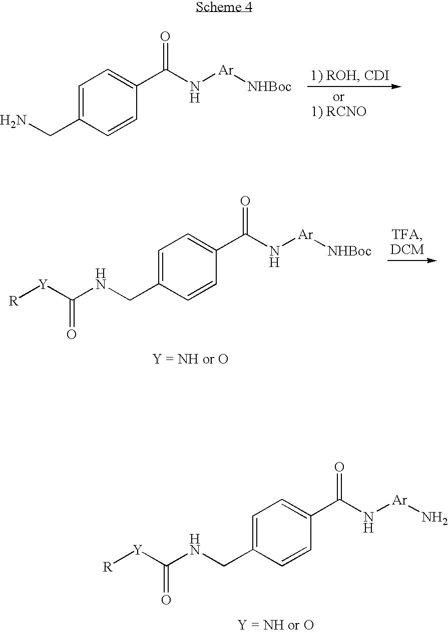

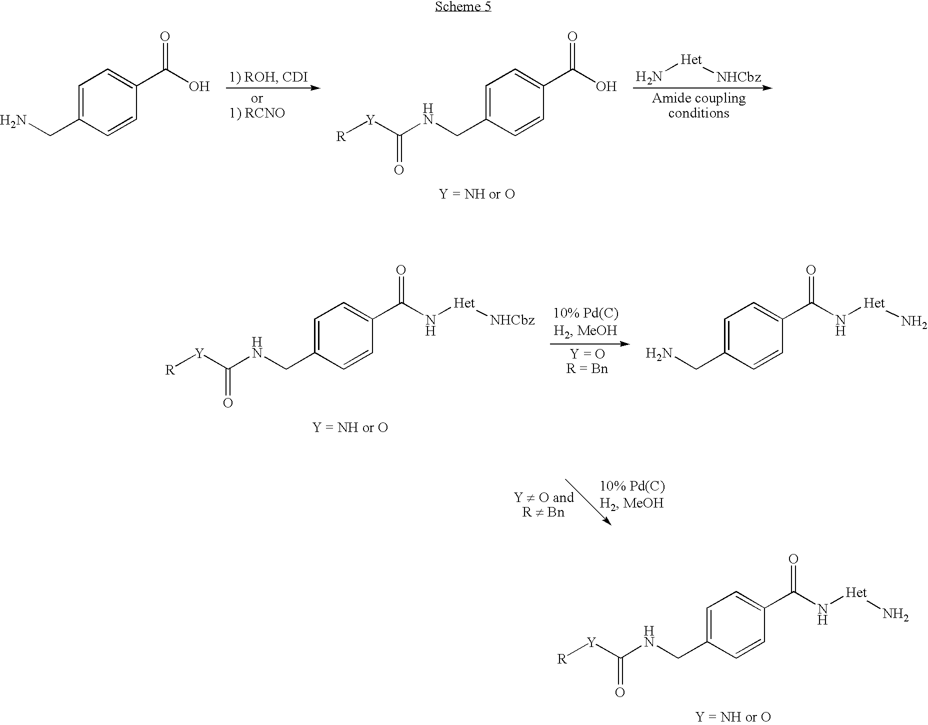

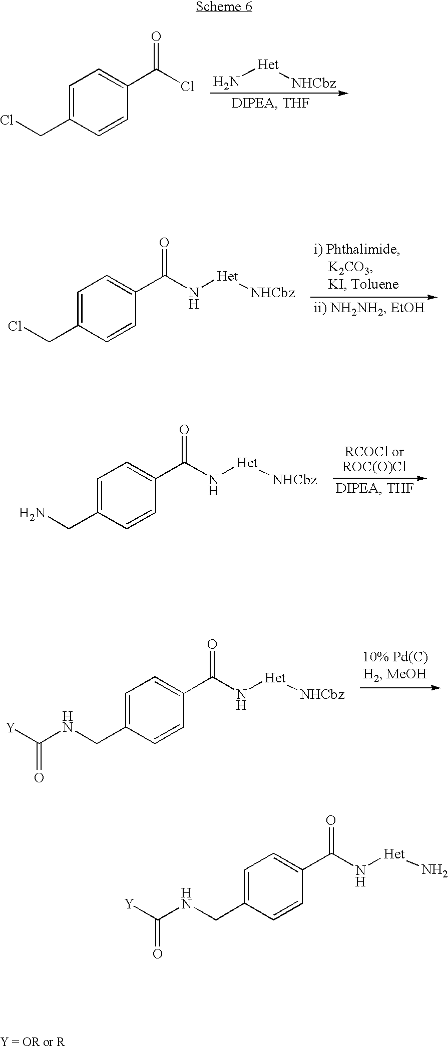

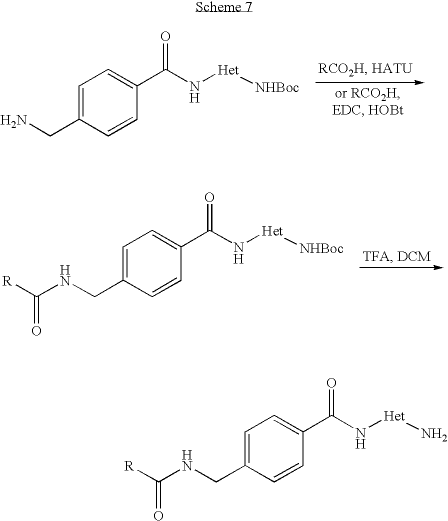

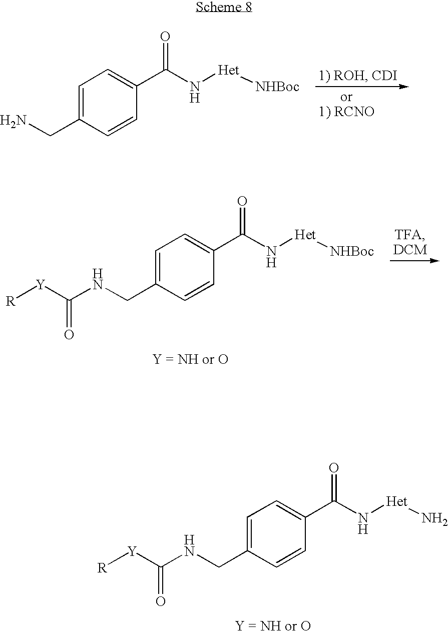

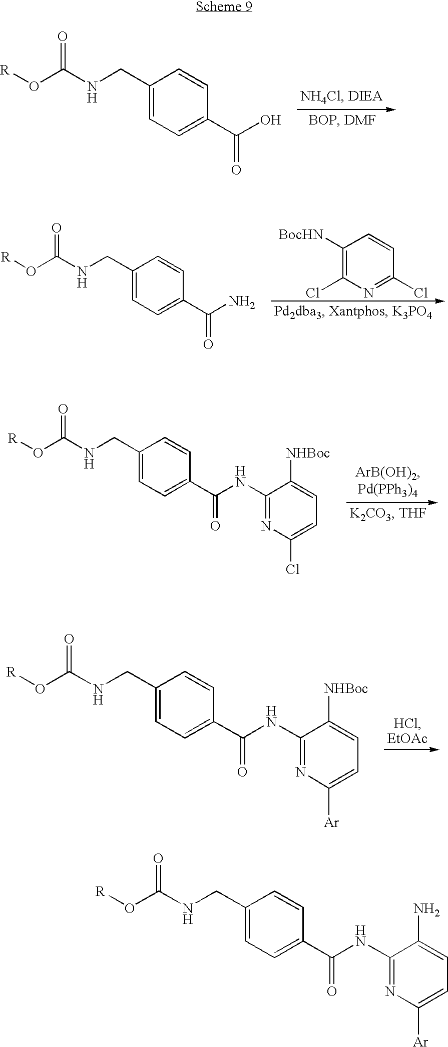

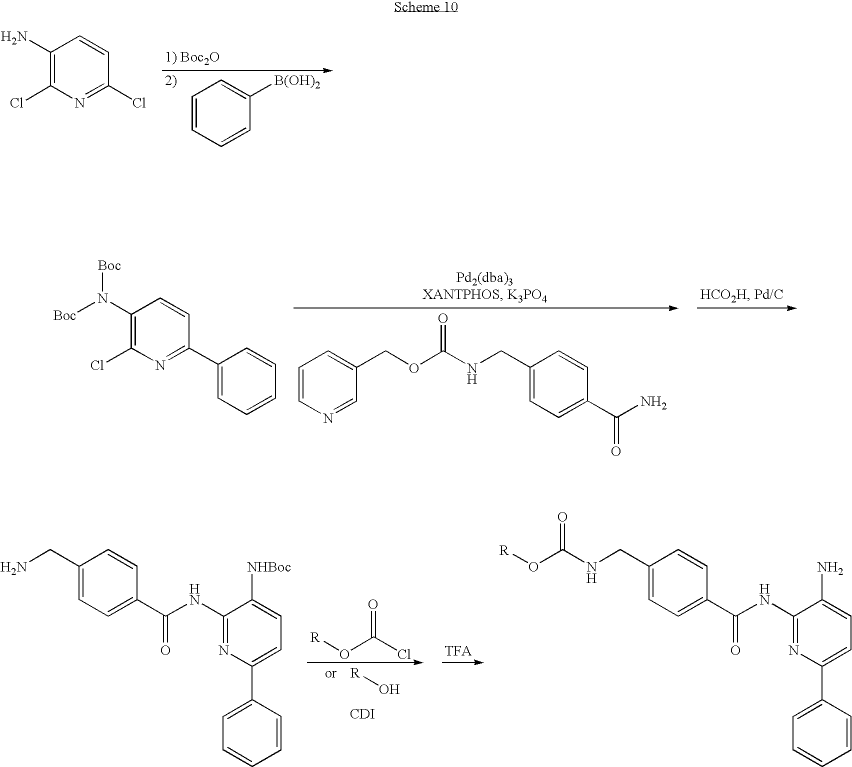

### Example 2

### Fluorescence Assays

Binding Studies Performed with the FITC-Labeled Compounds:

K_{d} Determination of FITC-Labeled Compounds

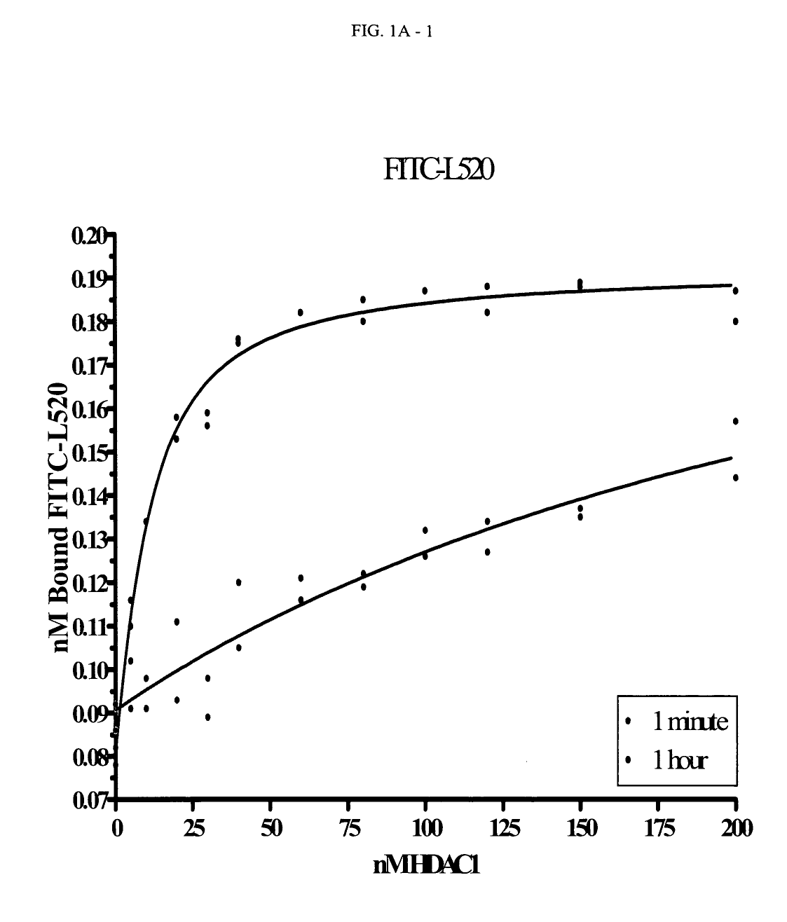

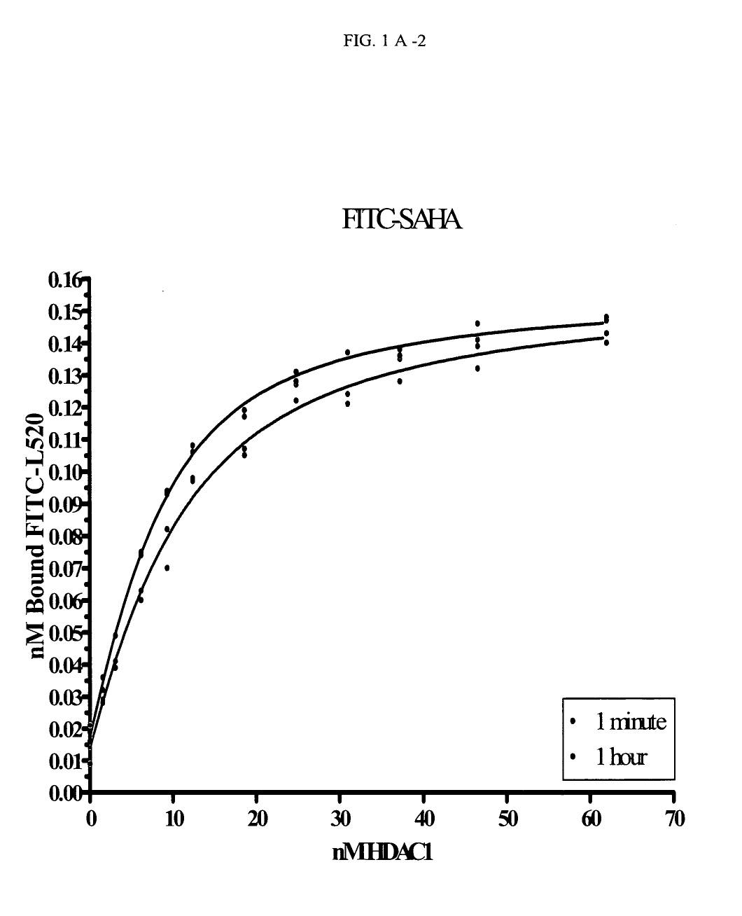

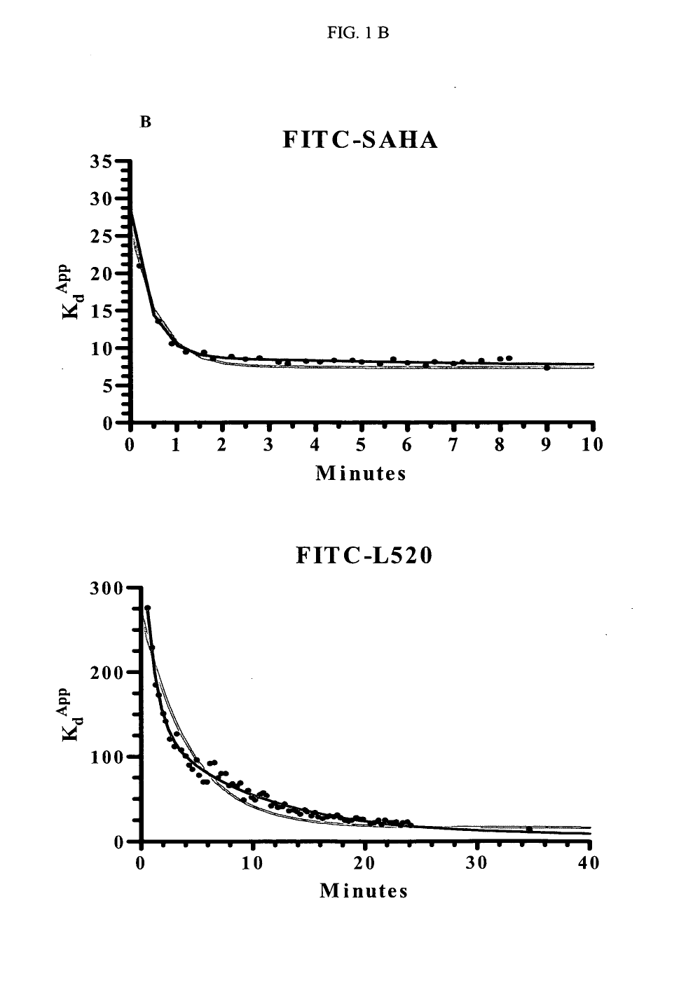

Titrations of HDAC1 were set up in 96-well black, flat-bottom plates. The concentrations of HDAC1 varied from 5 to 200 nM (with COMPOUND 1 and COMPOUND 2) or 20 to 450 nM (with COMPOUND 3). At time zero, the FITC-labeled compound was added and the plate inserted into the Analyst HT for FP detection. The samples were read every 5 seconds through ˜10 minutes (COMPOUND 1) or 2 hours (COMPOUND 2 and COMPOUND 3). The data at each time point was converted from mP to 1 nA units and plotted in Prism using an equation for ligand binding taking ligand-depletion into consideration (Equations 1-3) (An example of which is given in FIG. 1A).

$$\begin{matrix}C = {L}_{t} + {R}_{t} + {K}_{d} & ( Equation 1 ) \\ Bound = \frac{C}{2 {L}_{t}} - \frac{\sqrt{{C}^{2} - 4 {L}_{t} {R}_{t}}}{2 {L}_{t}} & ( Equation 2 ) \\ Signal ( Y ) = {A}_{f} + Bound * ( {A}_{b} - {A}_{f} ) & ( Equation 3 )\end{matrix}$$

where Lₜ is total fluorescently-labeled compound concentration, Rₜ is total HDAC concentration, K_{d} is the dissociation constant for the ligand to HDAC, A_{b} is the anisotropy when 100% of the ligand is bound, and A_{f} is the anisotropy when 100% of the ligand is free. The values determined by the data as plotted in Figure A are K_{d}^{app}, A_{b}, and A_{f}.

After determining the K_{d}^{app} for each time point, the K_{d}^{app} was replotted versus time (FIG. 1B) to determine true steady-state K_{d}. It was shown that COMPOUND 1 had a classical 1-step binding mechanism as it fit to a 1-exponential decay curve resulting in a true K_{d} of 16 nM. This binding came to equilibrium faster than could be measured by the instrument used. COMPOUND 2, however, fit better to a two-phase exponential curve (in blue) and slowly came to equilibrium over 210 minutes suggesting that this compound bound slowly with an induced-fit mechanism (see below). The plateau value of this exponential decay defines the steady-state K_{d} value.

The slow-type inhibitor binding consists of 2 steps: the first is a rapid-forming low affinity complex (EI); the second involves a slow conformational change of either the inhibitor or the enzyme to form a higher affinity complex (E*I). The SAHA-like inhibitor class demonstrates only the first, fast step. Kₘ and k_{cat} are the kinetic constants describing the conversion of substrate (S) to product (P).

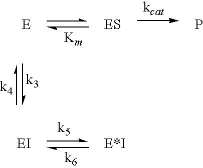

It was demonstrated that the affinities of the parental, unlabeled compounds were not altered by the addition of the FITC moiety.

### Competition Studies to Characterize Binding of the Unlabeled Compounds.

Flag-tagged HDAC1 (40 nM) was preincubated at RT for 1 hour with 2.5 nM FITC-labeled SAHA (COMPOUND 1) in a black, flat-bottomed 96-well plate. At time t=0, a titration of test compound from 0.3 nM to 5 μM was added to different wells and the dissociation of FITC-SAHA was monitored every minute for 3 hours using an Analyst HT plate reader. At each time point, the % FITC-SAHA bound to HDAC was calculated, plotted versus compound concentration, and a 4-parameter logistic fit was applied to the data to determine the inflection point. These inflection points were replotted versus time and the data fit to a 2-phase exponential decay. The plateau value of this decay gave the steady-state Kᵢ^{app,}* which was transformed into the steady-state Kᵢ* by the following equation taking into consideration ligand and receptor depletion:

$${K}_{i}^{*} = \frac{{K}_{i}^{* , app}}{1 + [ \frac{{L}_{T} + {R}_{T} + ( 1.5 ) B}{{K}_{D}} ]} \qquad ( Equation 4 )$$

Where, L_{T} is the total FITC-SAHA concentration, R_{T} the total HDAC concentration, B the concentration of the HDAC-FITC-SAHA complex, and K_{D} the dissociation constant of FITC-SAHA for HDAC.

### Determination of Kᵢ Values and Mechanism of Binding of Test Compounds.

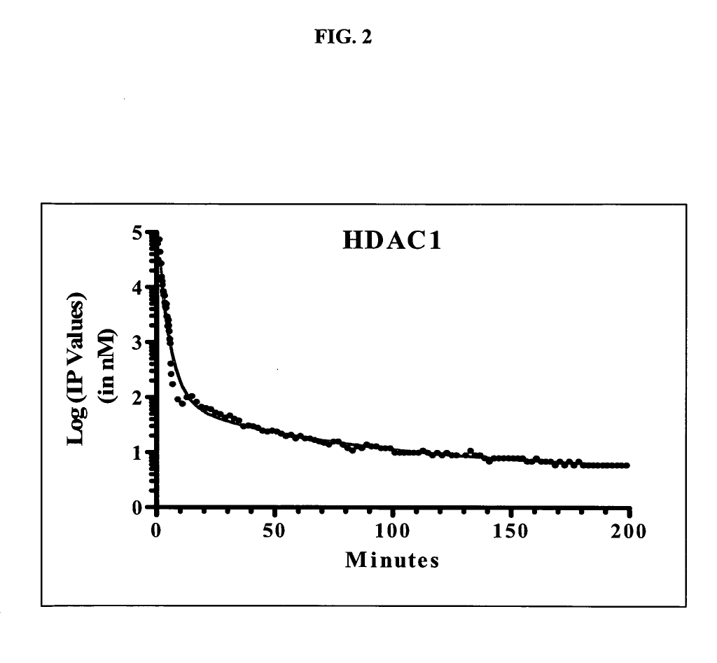

The IP value was determined every minute for 3 hours and replotted versus time (example given in FIG. 2). Some test compound data could not be fit to a single phase exponential decay curve which would be expected for a simple association-dissociation equilibrium. Rather, a 2-phase exponential decay curve had to be used to fit the data indicating a more complicated series of events was occurring during binding.

While this invention has been particularly shown and described with references to embodiments thereof, it will be understood by those skilled in the art that various changes in form and details may be made therein without departing from the meaning of the invention described. Rather, the scope of the invention is defined by the claims that follow.

What is claimed is:

1. A compound represented by the following structural Formula 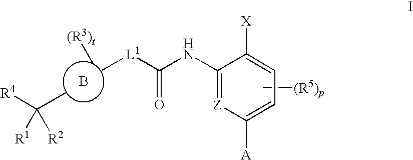
wherein A is aryl, heteroaryl or H, optionally substituted with halo, methyl, methoxy, amino, hydroxyl or halomethyl;
R¹ and R² are independently selected from H, OH, halo, NH₂, C₁-C₄ alkyl, or C₁-C₄ alkoxy;
R³ is independently selected from H, OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino;
R⁴ is selected from —NR⁶R⁷;
R⁵ is independently selected from H, OH, NH₂, nitro, CN, amide, carboxyl, C₁-C₂ alkoxy, C₁-C₂ alkyl, C₁-C₂ haloalkyl, C₁-C₂ haloalkyloxy, C₁-C₂ hydroxyalkyl, C₁-C₂ alkenyl, C₁-C₂ alkyl-C(═O)O—, C₁-C₂ alkyl-C(═O)—, C₁-C₂ alkynyl, halo, hydroxyalkoxy, C₁-C₂ alkyl-NHSO₂—, C₁-C₂ alkyl-SO₂NH—, C₁-C₂ alkylsulfonyl, C₁-C₂ alkylamino or di(C₁-C₂)alkylamino;
R⁶ is independently selected from H or C₁-C₄ alkyl;
R⁷ is selected from —(CR^{a}₂)ₛC(O)(CR^{a}₂)_{q}R¹², or —(CR^{a}₂)C(O)O(CR^{a}₂)_{q}R¹²;
R¹² is selected from C₁-C₄ alkyl, C₃-C₆ cycloalkyl, heteroaryl, aryl or heterocyclic, wherein the alkyl, cycloalkyl, heteroaryl, heterocyclic or aryl is attached to a fluorophore through a linker, and optionally substituted OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino, aryl, heterocyclic or cycloalkyl;
R^{a} is independently selected from H or C₁-C₄ alkyl;
p is 1, 2, 3 or 4;
s and q are independently 0, 1, 2, 3, or 4;
L¹ is (CH₂)ᵣ, ethenyl or cyclopropyl, wherein r is 0, 1 or 2;
X is OH or NH₂;
Z is C or N;
or a stereoisomer or pharmaceutically acceptable salt thereof.

2. The compound of claim 1, wherein
A is 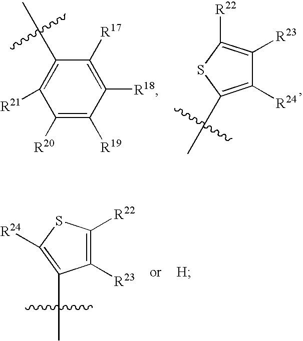
R¹ and R² are independently selected from H, OH, halo, NH₂, C₁-C₄ alkyl, or C₁-C₄ alkoxy;
R³ is H;
R⁴ is —NR⁶R⁷;
R⁵ is H;
R⁶ is selected from H or C₁-C₄ alkyl;
R⁷ is —C(O)O(CR^{a}₂)_{q}R¹²;
R¹² is selected from aryl or heteroaryl; wherein the aryl or heteroaryl is attached to a fluorophore through a linker, and optionally substituted with OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-SO₂NH—, C₁-C₇ alkylsulfonyl, C₁-C₇ alkylamino or di(C₁-C₇)alkylamino, aryl, heterocyclic or cycloalkyl;
R¹⁷ and R²¹ are independently selected from hydrogen or fluoro;
R¹⁸, R¹⁹ or R²⁰ are independently selected from hydrogen, halo, methyl, methoxy or halomethyl;
R²², R²³ and R²⁴ are independently selected from hydrogen, methyl, amino, hydroxyl or halo;
R^{a} is independently H or C₁-C₄ alkyl;
Ring B is aryl or heteroaryl;
q is independently 0, 1 or 2;
L¹ is a bond;
X is NH₂;
or a stereoisomer or pharmaceutically acceptable salt thereof.

3. The compound of claim 2, wherein A is

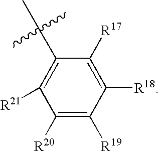

4. The compound of claim 2, wherein R¹ and R² are H; R^{a} is H; R⁶ is H, and q is 1.

5. The compound of claim 1, wherein the fluorophore is selected from the fluorophore in fluorescein, BODIPY TMR dye, BODIPY TR dye, Cascade Blue, Cascade Yellow, Dapoxyl Dyes, Marina Blue, Lucifer yellow, Pacific Blue dyes, Oregon Green 488 dye, Oregon Green 514 dye, NODIPY FL dye, tetramethylrhodamine, rhodamine, X-Rhodamine, rhodamine 6G, rhodamine B, rhodamine 123, Rhodamine Red, Rhodamine Green, Rhodol Green, sulforhodamine 101, Texas Red, coumarin, hydroxycoumarin, aminocoumarin, methoxycoumarin, cyanine, Alexa Fluor dyes, DyLight 549 and DyLight 633.

6. The compound of claim 1, wherein the linker is 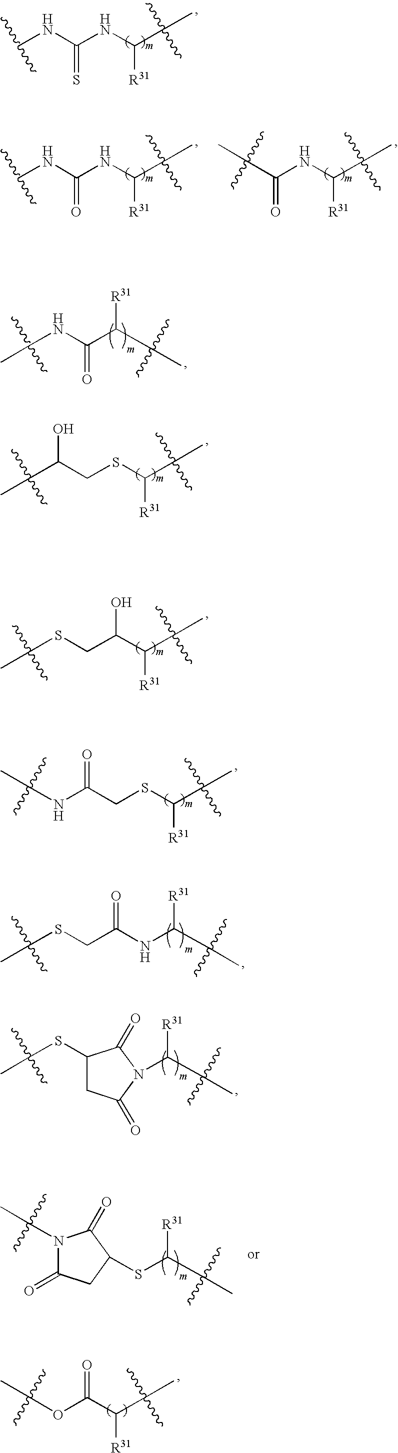
wherein R³¹ is H or C₁-C₄ alkyl;
m is 0, 1 or 2.

7. A compound represented by the following structural Formula 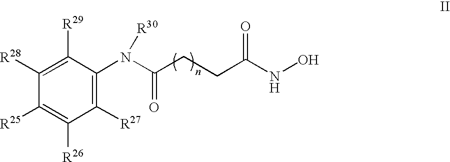
Wherein,
R²⁵ is F-L-, where L is a linker, and F is a fluorophore;
R²⁶ to R²⁹ is independently selected from H, C₁-C₄ alkyl, CN, azido, C₁-C₄ cyanoalkyl, nitro, halo, C₁-C₄ haloalkyl, amino, amide, carboxyl, C₁-C₄ alkoxycarbonyl, C₁-C₄ alkylaminocarbonyl, hydroxyl, C₁-C₄ alkoxy, aryl-C₁-C₄-alkoxy, C₁-C₄ haloalkyloxy, C₁-C₄ hydroxyalkyl, C₁-C₄ alkenyl, C₁-C₄ alkyl-C(═O)O—, C₁-C₄ alkyl-C(═O)—, C₁-C₄ alkynyl, hydroxyalkoxy, C₁-C₄ alkyl-NHSO₂—, C₁-C₄ alkyl-SO₂NH—, C₁-C₄ alkylsulfonyl, C₁-C₄ alkylamino or di(C₁-C₄)alkylamino;
R³⁰ is selected from H or C₁-C₄ alkyl;
n is 4, 5, 6, 7 or 8;
or a stereoisomer or pharmaceutically acceptable salt thereof.

8. The compound of claim 7, wherein the fluorophore is selected from the fluorophore in fluorescein, BODIPY TMR dye, BODIPY TR dye, Cascade Blue, Cascade Yellow, Dapoxyl Dyes, Marina Blue, Lucifer yellow, Pacific Blue dyes, Oregon Green 488 dye, Oregon Green 514 dye, NODIPY FL dye, tetramethylrhodamine, rhodamine, X-Rhodamine, rhodamine 6G, rhodamine B, rhodamine 123, Rhodamine Red, Rhodamine Green, Rhodol Green, sulforhodamine 101, Texas Red, coumarin, hydroxycoumarin, aminocoumamm, methoxycoumarin, cyanine, Alexa Fluor dyes, DyLight 549 and DyLight 633.

9. The compound of claim 7, wherein the linker is 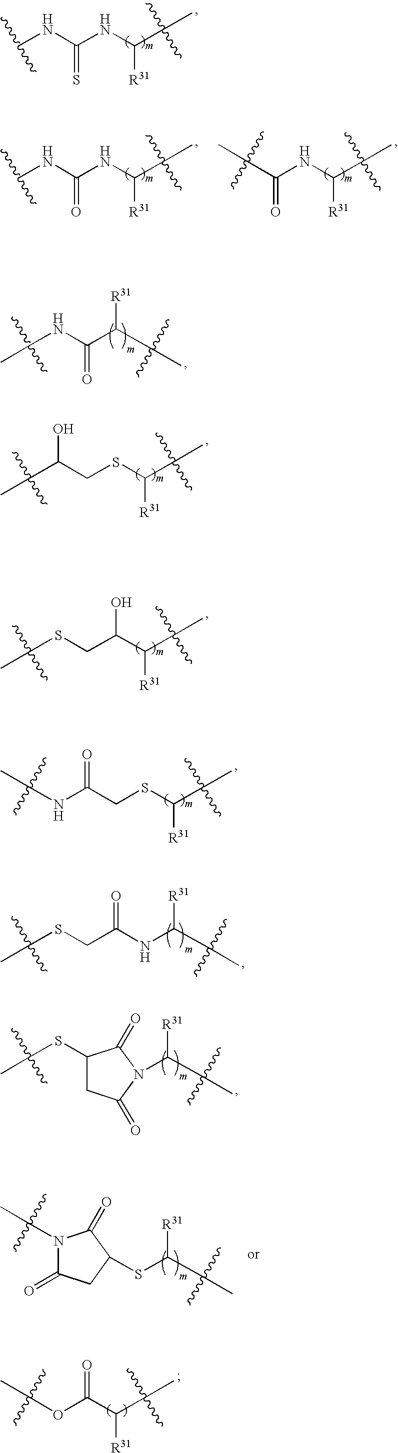
wherein R³¹ is H or C₁-C₄ alkyl;
m is 0, 1 or 2.

10. A compound selected from:
5-{[({4-[({[(4-{[(4-aminobiphenyl-3-yl)amino]carbonyl}benzyl)amino]carbonyl}oxy)methyl]benzyl}amino)carbonothioyl]amino}-2-(6-hydroxy-3-oxo-3H-xanthen-9-yl)benzoic acid;
5-({[(4-{[8-(hydroxyamino)-8-oxooctanoyl]amino}benzyl)amino]carbonothioyl}amino)-2-(6-hydroxy-3-oxo-3H-xanthen-9-yl)benzoic acid;
5-{[({4-[({[(4-{[(2-aminophenyl)amino]carbonyl}benzyl)amino]carbonyl}oxy)methyl]benzyl}amino)carbonothioyl]amino}-2-(6-hydroxy-3-oxo-3H-xanthen-9-yl)benzoic acid.
or a stereoisomer or pharmaceutically acceptable salt thereof.
